(19) United States
(12) Patent Application Publication
(10) Pub. No.: US 2010/0311821 A1
(43) Pub. Date: Dec. 9, 2010

(54) Synthetic vector

(76) Inventor: Yan Geng, Athens, GA (US)

Correspondence Address: MUETING, RAASCH & GEBHARDT, P.A., P.O. BOX 581336, MINNEAPOLIS, MN, 55458-1336, US

(21) Appl. No.: 12/798,996

(22) Filed: Apr. 15, 2010

Related U.S. Application Data
(60) Provisional application No. 61/169,400, filed on Apr. 15, 2009.

(51) Int. Cl. A61K 31/711; C12N 5/071; A61P 43/00

(52) U.S. Cl. 514/44 R; 435/325

(57) ABSTRACT

The present invention includes compositions including a polymer-peptide hybrid and one or more isolated polynucleotides, wherein the polymer of the polymer-peptide hybrid includes a neutral amphiphilic block copolymer, wherein the peptide of the polymer-peptide hybrid is covalently attached to the hydrophobic block of the amphiphilic block copolymer, and wherein the one or more isolated polynucleotides are complexed with the polymer-peptide hybrid via electrostatic interactions between the peptide of the polymer-peptide hybrid and the one or more polynucleotides. The present invention also includes methods of making and using such compositions.

### CONTINUING APPLICATION DATA

This application claims the benefit of U.S. Provisional Application Ser. No. 61/169,400, filed Apr. 15, 2009, which is incorporated by reference herein in its entirety.

### BACKGROUND

The promise of human gene technology in advancing science and medicine is well recognized and vigorously pursued. While genetically manipulating bacteria and primitive eukaryotic cells, such as, for example, yeast, is well-established, the next level of genetic technology on human cells in the applications of gene therapy, as well as cell reprogramming and tissue engineering, is facing far more complicated challenges. See, for example, R. C. Mulligan, Gene Transfer and Gene Therapy: Principles, Prospects and Perspective, in Etiology of Human Disease at the DNA Level, ed. J. Lindsten and U. Pettersson, Raven Press, New York, 1991, pp. 143-189; Takahashi et al., 2007, Cell; 131:861-872; and Bonadio, 2000, J. Mol. Med.; 78:303-311. Among the various challenging aspects, the development of a safe and efficient gene transfer vector that can effectively package and protect genes and transport them across barriers into the cell nucleus is a bottleneck (Lehn et al., 1998, Adv. Drug Delivery Rev.; 30:5-11). Naked genes can not enter cells on their own, and are easily degraded by nucleases in biofluids. The conventional mechanical transfection techniques, such as, for example, electroporation, that work well on bacteria and yeasts, tend to cause poor viability in human cells (Somiari, 2000, Mol. Ther.; 2:178-187). Transfection techniques by the use of recombinant viral vectors, though highly efficient, are associated with innate immunogenicity and safety issues that are critical concerns in human gene technology (Lehn et al., 1998, Adv. Drug Delivery Rev.; 30:5-11). Unlike viral vectors, synthetic vectors are free of these risks. The development of synthetic vectors is still at a very early stage. And, significant knowledge gaps exist in various aspects of synthetic vector design, such as, for example, vector-gene complex formation, dissociation (i.e. gene release) mechanism, and biological interactions of the vector-gene complex with the host from transport, through internalization to transfection. Thus, there is a need for improved synthetic vectors.

### SUMMARY OF THE INVENTION

The present invention includes a composition that includes a polymer-peptide hybrid and one or more isolated polynucleotides; wherein the polymer of the polymer-peptide hybrid includes a neutral amphiphilic block copolymer; wherein the peptide of the polymer-peptide hybrid is covalently attached to the hydrophobic block of the amphiphilic block copolymer; and wherein the one or more isolated polynucleotides are complexed with the polymer-peptide hybrid via electrostatic interactions between the peptide of the polymer-peptide hybrid and the one or more polynucleotides.

In some aspects of a composition of the present invention, an amphiphilic block copolymer includes a diblock copolymer.

In some aspects of a composition of the present invention, the hydrophobic block of the amphiphilic block copolymer includes one or more double bonds available for free radical addition reaction of a thiol group of the peptide.

In some aspects of a composition of the present invention, the hydrophilic block of the amphiphilic block copolymer includes polyethylene glycol (PEG) or poly(ethylene oxide) (PEO).

In some aspects of a composition of the present invention, the hydrophobic block of the amphiphilic block copolymer includes a hydrophobic block selected from polybutadiene (PBD), polyethyl ethylene (PEE), poly(2-isopropyl-2-oxazoline), and combinations thereof.

In some aspects of a composition of the present invention, the amphiphilic block copolymer includes poly(ethylene glycol)-block-polybutadiene (PEG-b-PBD). In some aspects, PBD-b-PEG includes PBDₘ-b-PEGₙ, wherein n and m are independently about 8 to about 120. In some aspects, PBD-b-PEG includes PBD₁₄-b-PEG₉₃ or PBD₂₅-b-PEG₇₅.

In some aspects of a composition of the present invention, the peptide includes Kₙ, Rₙ, KₙW, RₙW, KWKₙ₋₁, RWRₙ₋₁, KₙH, RₙH, KHKₙ₋₁, RHRₙ₋₁, KₙP, RₙP, KPKₙ₋₁, or RPRₙ₋₁, wherein n=1, 2, 3, 4, 5, 6, 7, 8, 9, or 10. In some aspects, the peptide includes KWK, KWK₂, K₂WK, KWK₄, or K₄WK.

In some aspects of a composition of the present invention, the grafting density of the peptide to the hydrophobic block of the amphiphilic block copolymer is about 1 to about 10. In some aspects, the grafting density of the peptide to the hydrophobic block of the amphiphilic block copolymer is about 4, about 8, or about 12.

In some aspects of a composition of the present invention, one or more polynucleotide encode a protein product.

In some aspects of a composition of the present invention, the one or more isolated polynucleotides condense into a compact, ordered DNA condensate. In some aspects, the DNA condensate forms a rod structure, a toroid structure, and/or a spherical structure.

In some aspects, a composition of the present invention further includes a pharmaceutical carrier suitable for administration to a mammal for gene therapy.

The present invention includes methods of delivering one or more isolated polynucleotides to a cell, the method comprising contacting the cell with a composition of the present invention.

The present invention includes methods of delivering one or more isolated polynucleotides to a subject, the method comprising administering a composition of the present invention to the subject.

The present invention includes a polymer-peptide hybrid; wherein the polymer of the polymer-peptide hybrid includes a neutral amphiphilic block copolymer; wherein the peptide of the polymer-peptide hybrid is covalently attached to the hydrophobic block of the amphiphilic block copolymer; and wherein the peptide of the polymer-peptide hybrid is a DNA binding peptide.

In some aspects of the polymer-peptide hybrid of the present invention, an amphiphilic block copolymer includes a diblock copolymer.

In some aspects of the polymer-peptide hybrid of the present invention, the hydrophobic block of the amphiphilic block copolymer includes one or more double bonds available for free radical addition reaction of a thiol group of the peptide.

In some aspects of the polymer-peptide hybrid of the present invention, the hydrophilic block of the amphiphilic block copolymer includes polyethylene glycol (PEG) or poly(ethylene oxide) (PEO).

In some aspects of the polymer-peptide hybrid of the present invention, the hydrophobic block of the amphiphilic block copolymer includes a hydrophobic block selected from polybutadiene (PBD), polyethyl ethylene (PEE), poly(2-isopropyl-2-oxazoline), and combinations thereof.

In some aspects of the polymer-peptide hybrid of the present invention, the amphiphilic block copolymer includes poly(ethylene glycol)-block-polybutadiene (PEG-b-PBD). In some aspects, PBD-b-PEG includes PBDₘ-b-PEGₙ, wherein n and m are independently about 8 to about 120. In some aspects, PBD-b-PEG includes PBD₁₄-b-PEG₉₃ or PBD₂₅-b-PEG₇₅.

In some aspects of the polymer-peptide hybrid of the present invention, the peptide includes Kₙ, Rₙ, KₙW, RₙW, KWKₙ₋₁, RWRₙ₋₁, KₙH, RₙH, KHKₙ₋₁, RHRₙ₋₁, KₙP, RₙP, KPKₙ₋₁, or RPRₙ₋₁, wherein n=1, 2, 3, 4, 5, 6, 7, 8, 9, or 10. In some aspects, the peptide includes KWK, KWK₂, K₂WK, KWK₄, or K₄WK.

In some aspects of the polymer-peptide hybrid of the present invention, the grafting density of the peptide to the hydrophobic block of the amphiphilic block copolymer is about 1 to about 10. In some aspects, the grafting density of the peptide to the hydrophobic block of the amphiphilic block copolymer is about 4, about 8, or about 12.

The present invention includes compositions of the polymer-peptide hybrid of the present invention. In some aspects, the composition further includes one or more polynucleotides. In some aspects, the one or more polynucleotide encode a protein product. In some aspects, the composition further includes a pharmaceutical carrier suitable for administration to a mammal.

The present invention includes methods of administering a polymer-peptide hybrid of the present invention to a subject and methods of contacting a cell with a polymer-peptide hybrid of the present invention.

The terms “comprises” and variations thereof do not have a limiting meaning where these terms appear in the description and claims.

Unless otherwise specified, “a,” “an,” “the,” and “at least one” are used interchangeably and mean one or more than one.

### BRIEF DESCRIPTION OF THE FIGURES

FIG. 1 is a schematic illustration of combinative polymer-peptide hybrids self-assembling with genes into compact nanostructures.

FIGS. 2A-2B present schematic representation (FIG. 2A) and mechanism (FIG. 2B) for grafting Cys-oligopeptide onto PEG-b-PBD via free radical addition.

FIG. 3 demonstrates change of the morphology towards lower curvature structures through the attachment of a hydrophobic peptide onto PBD-b-PEO.

FIG. 4A represents a possible reaction pathway of the intermediate radicals formed by the addition of RS onto PBD. FIG. 4B presents the chemical structure of C- and CF-grafted PBD-b-PEO.

FIG. 5A presents 1^{H} NMR spectrum (solvent: [D₆]DMSO). FIG. 6B presents SEC (eluent: NMP; solid line: RI; dashed line: UV, λ=270 nm) of 1-CF.

FIG. 6 presents a visualization of self-assembled structures of PBD-b-PEO (panels 1 and 2) and C-grafted hybrids (panels 1-C and 2-C) by cryo-TEM (scale bars=50 nm) and CF-grafted hybrids (panels 1-CF and 2-CF) by FM (scale bars=5 μm) in water.

FIG. 7A presents FM snapshots of I) right-handed and ii) left-handed distorted helical structures (scale bars=2 μm). FIG. 7B presents CD spectrum of 1-CF wormlike micelles in water.

FIGS. 8A-8C represent AFM imaging in 10 mM sodium cacodylate buffer of KWK-DNA complex (FIG. 8A), PP4-DNA complex (FIG. 8B), and PP8-DNA complex (FIG. 8C). In FIG. 8A, the arrow points to an individual ds-λDNA strand protruding from the bundle.

FIG. 9 presents melting profiles of 50 μg ml⁻¹ native λ-DNA (a), DNA-KWK complex (b), DNA-PP4 complex (c), and DNA-PP8 complex (d) in 10 mM sodium cacodylate buffer.

FIG. 10 is a schematic representation of combinative self-assembly of the block copolymer-peptide clustered hybrid with DNA into toroid and rod condensates.

FIG. 11 is a characterization of the binding and conformational change of λ-DNA complexation with KWK₂, PP12 and PP24. FIG. 11A presents EB displacement assay on DNA binding. FIG. 11B presents DNA conformation by CD analysis. FIG. 11C is representative TEM micrographs of λ-DNA condensates by KWK₂, PP12 and PP24 at N/P=12. Scale bar is 50 nm.

FIG. 12 is a characterization of the binding and conformational change of λ-DNA complexation with KWK₄, PP20 and PP40. FIG. 12A presents EB displacement assay on DNA binding. FIG. 12B presents DNA conformation by CD analysis. FIG. 12C is representative TEM micrographs of λ-DNA condensates by KWK₄, PP20 and PP40 at increasing N/P from 3 to 12. Scale bar is 50 nm.

FIG. 13 presents stability of DNA complexes against thermal and bio-degradation. FIG. 13A is melting profiles of native λ-DNA and its complexes with KWK₄, PP20 and PP40. FIG. 13B is a table of estimated Tm of native λ-DNA and its complexes with KWK₄, PP20 and PP40. FIG. 13C is results of Dnasel protection assay. (−) before treatment, (+) after treatment.

FIG. 14 is a schematic representation providing definitions of diameter and thickness of a toroid.

FIG. 15 is a schematic representing the grafting of Cys-oligopeptides into the PEG-b-PBD via free radical addition route.

FIG. 16 is characterization of the polymer-peptide hybrid by ¹H NMR and GPC. FIG. 16A is a representative ¹H NMR of the polymer-peptide hybrid (solvent: DMSO-d2 of PP40). FIG. 16B is a representative GPC (eluent: NMP) of PEG-b-PBD precursor (solid line) and PP40 (dashed line).

FIG. 17 is a schematic of condensation and packaging of various forms of DNA via the combinative self-assembly. FIG. 17A is double-stranded linear, supercoiled, relax-circular. FIG. 17B is single-stranded linear, circular.

FIG. 18 presents AFM imaging (FIG. 18A) and agarose gel electrophoresis (FIG. 18B) of various forms of FX174 DNA. In FIG. 18B lane 1 is ds linear; lane 2 is ds sc; lane 3 is ds rc; lane 4 is ss linear; and lane 5 is ss circular.

FIG. 19 is representative TEM images of the five different forms of FX174 DNA condensates via the combinative self-assembly.

FIG. 20 presents binding of PP40 with ΦX174 DNA's via EB Displacement Assay. FIG. 20A is ds ΦX174 DNA. FIG. 20B is ss ΦX174. I₀=fluorescence intensity of DNA-EB complex without addition of PP40. For each form of DNA, PP40 continuously binds to DNA and quenches the fluorescence as N/P increases. Upon N/P=3, PP40 is able to achieve nearly complete quenching (I/I₀≦0.3), indicating complete binding between PP40 and DNA.

### DETAILED DESCRIPTION OF ILLUSTRATIVE EMBODIMENTS OF THE INVENTION

The present invention provides improved synthetic vectors and methods of making and using such synthetic vectors. The synthetic vectors of the present invention have utility in a variety of pharmaceutical and biotechnology applications. For example, the vectors of the present invention provide improved vectors systems for gene therapy to fight serious diseases, such as cancer, for the delivery of differentiation signals in stem cell therapies, and for improved vaccines.

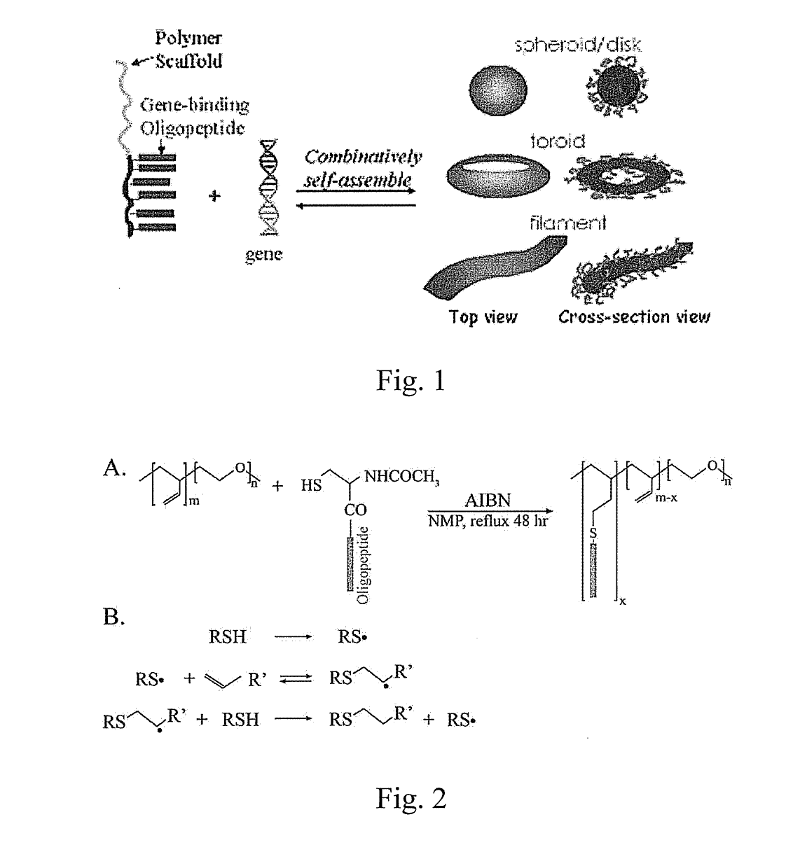

The synthetic vectors of present invention are compositions that include a polymer-peptide hybrid and one or more isolated polynucleotides. The polymer of the polymer-peptide hybrid may be an amphiphilic block copolymer, including, but not limited to, a neutral amphiphilic block copolymer. The peptide of the polymer-peptide hybrid is covalently attached to the hydrophobic block of the amphiphilic block copolymer. And, the one or more isolated polynucleotides may be complexed with the polymer-peptide hybrid via electrostatic interactions between the peptide of the polymer-peptide hybrid and the one or more polynucleotides. A schematic illustration of the synthetic vectors of the present invention is shown in FIG. 1.

The synthetic vector compositions of the present invention include, as one component, a polymer-peptide hybrid. With the present invention, block copolymers serve as a polymer scaffold or scaffolding to which peptides are covalently attached. Block copolymers are made up of blocks of different polymerized monomers. The block copolymers of the present invention include two or more homopolymer subunits linked by covalent bonds and contain long contiguous blocks of two or more repeating units in the same polymer chain. As used herein, “polymers” are macromolecules having connected monomeric units. In a block copolymer, instead of a mixed distribution of monomeric units, a long sequence or block of one monomer is joined to a block of the second monomer. Block copolymers are polymers having at least two, tandem, interconnected regions of differing chemistry. Each region comprises a repeating sequence of monomers. Thus, a “diblock copolymer” comprises two such connected regions (A-B); a “triblock copolymer,” three (A-B-C), etc. The present invention includes block copolymers with two, three, four, or more distinct blocks, called diblock copolymers, triblock copolymers, tetrablock copolymers, and multiblock copolymers, respectively.

In referring to a block copolymer, one may insert the letter “b” between the identifiers for the respective polymer blocks, such as A-b-B. For example, “PEG-b-PBD” is a block copolymer of poly(ethylene glycol) (PEG) homopolymer subunits and poly(butadiene) (PBD) homopolymer subunits. In referring to a block copolymer, one may insert a subscript after an identifier for a polymer block, to indicate the average number of monomeric units in a block, such as, for example, Aₘ-b-Bₙ.

The block copolymer of the polymer-peptide hybrid may be an amphiphilic block copolymer, including, but not limited to, a neutral amphiphilic block copolymer. As used herein, an “amphiphilic block copolymer” is a block copolymer having at least one hydrophilic (water-soluble, polar) polymer chain and at least one hydrophobic (water-insoluble) polymer chain which are covalently linked. See, for example, Mecke et al., 2006, Soft Matter; 2:751-759.

Block copolymers can be made by any of a variety of known methods, including, for example, condensation reactions and living polymerization techniques, such as, for example, atom transfer free radical polymerization (ATRP), reversible addition fragmentation chain transfer (RAFT), ring-opening metathesis polymerization (ROMP), living cationic or living anionic polymerizations, and chain shuttling polymerization. Diblock copolymers may be prepared, for example, using a two-step anionic polymerization procedure (Hillmyer et al., 1996, Macromolecules, 29:6994-7002), wherein copolymers are dissolved in chloroform and dried on glass to form a film that is hydrated with water at 50-60° C. Amphiphilic block copolymers of the present invention include, but are not limited to, any of the amphiphilic block copolymers presented in U.S. Pat. Nos. 6,835,394 and 7,217,427 and U.S. Patent Application Serial Nos. 2005 0180922 A1, 2006 0165810 A1, and 2007 0218123 A1, and 2008 0181939 A1.

With the present invention, the hydrophobic block of an amphiphilic block copolymer may include one or more double bonds utilized for the covalent attachment of the peptide to the hydrophobic block of the amphiphilic block copolymer, for example by a free radical addition reaction of a thiol group of the peptide. Hydrophobic blocks of the amphiphilic block copolymer of the present invention include any known hydrophobic monomer block, including, but not limited to, poly-2-methyloxazoline (PMOXA), polybutadiene (PBD), polyethylethylene (PEE), poly(2-isopropyl-2-oxazoline), or combinations thereof.

Hydrophilic blocks of the amphiphilic block copolymer of the present invention include any known hydrophilic monomer blocks, including, but not limited to, polyethylene glycol (PEG) and ionic polymers, such as polyacrylic acid (PAA). As used herein, polyethylene glycol (PEG) is a polymer of ethylene oxide also known as polyethylene oxide (PEO) or polyoxyethylene (POE). Thus, as used herein, PEG, PEO and POE are chemically synonymous.

In some embodiments, an amphiphilic block copolymer of the present invention includes polyethylene glycol (PEG) as the hydrophilic block and polybutadiene (PBD) as the hydrophobic block. That is, the amphiphilic block copolymer is poly(ethylene glycol)-block-polybutadiene (also referred to herein as “PBD-b-PEG” or “PEG-b-PBD”). In some embodiments, the amphiphilic block copolymer is PBDₙ, b-PEGₙ In some embodiments, each of n and m may be independently about 8 to about 120. For example, n may be about 8, about 10, about 14, about 20, about 25, about 30, about 40, about 50, about 60, about 70, about 75, about 80, about 90, about 100, about 110, or about 120, and m may be about 8, about 10, about 14, about 20, about 25, about 30, about 40, about 50, about 60, about 70, about 75, about 80, about 90, about 100, about 110, or about 120. In some embodiments, PBD-b-PEO comprises PBD₁₄-b-PEO₉₃ or PBD₂₅-b-PEO₇₅.

With the polymer-peptide hybrids of the present invention the peptide of a polymer-peptide hybrid is covalently attached to the hydrophobic block of the amphiphilic block copolymer. As used herein, a “peptide” is a linear chain of amino acids formed by the covalent linkage of amino acids via a peptide bond, also know as an amide bond. Peptides of the present invention may be synthesized using any of a variety of techniques known in the art. Alternatively, peptides are available for purchase from a variety of commercial suppliers.

With the present invention, the peptide of the polymer-peptide hybrid is covalently attached to the hydrophobic block of the amphiphilic block copolymer. For example, following procedures described in more detail in the examples section, the thiol group of a terminal cysteine reacts with a double bond of the hydrophobic block by the free radical addition reaction and clicks the oligopeptide into the amphiphilic block copolymer of the present invention (see FIGS. 2A and 2B). A peptide may be covalently attached via the thiol group of a cysteine amino acid residue of the peptide. Such a cysteine residues may be in a N-terminal position, a C-terminal position, or in an internal region of the peptide (between the N-terminus and the C-terminus). In some embodiments, covalent attachment may be via carboxyl groups within the peptide sequence or amino groups within the peptide sequence. With the present invention, the grafting density of the peptides to the block copolymer scaffold may be varied. For example, a grafting density of about 4, about 8, about 12, or more may be obtained.

A peptide of the present invention may be any of a wide variety of lengths. For example, a peptide of the present invention may be a dipeptide, tripeptide, tetrapeptide, a pentapeptide, a hexapeptide, a heptapeptide, an octapeptide, a nonapeptide, or a decapeptide, wherein a dipeptide has two amino acid residues; a tripeptide has three amino acids; a tetrapeptide has four amino acids; a pentapeptide has five amino acids; a hexapeptide has six amino acids; a heptapeptide has seven amino acids; an octapeptide has eight amino acids; a nonapeptide has nine amino acids; and a decapeptide has ten amino acids. A peptide of the present invention may be about ten or fewer amino acids in length. A peptide of the present invention may be less than five, six, seven, eight, nine, or ten amino acids in length. A peptide of the present invention may be five or fewer, six or fewer, seven or fewer, eight or fewer, nine or fewer, or ten or fewer amino acids in length. A peptide of the present invention may be greater than two, greater than three, or greater than four amino acids in length. A peptide of the present invention may be less than about 25 amino acids in length, less than about 30 amino acids long, less than about 40 amino acids in length, or less than about 50 amino acids in length. A peptide of the present invention may be 25 or fewer amino acids in length, 30 or fewer amino acids in length, 40 or fewer amino acids in length, or 50 or fewer amino acids in length. A peptide of the present invention have a size ranges of two or more of the above discussed sizes. For example, a peptide of the present invention may be three to 50 amino acids in length. A peptide of the present invention may be two to ten amino acids in length. A peptide of the present invention may be three to ten amino acids in length.

A peptide of the present invention may be a DNA binding peptide, including, but not limited to, oligopeptides that emulate active nucleotide binding sites for DNA condensation (see, for example, Sparrow et al., 1998, Adv Drug Deliv Rev; 30:115-131). DNA binding peptides may bind with DNA via various interactions, including, for example, electrostatic interactions between positively charged amino acids, such as lysine (“Lys” or “K”) or arginine (“Arg” or “R”), with the negatively charged phosphate DNA backbone, and the intercalation of aromatic amino acids, such as tryptophan (“Trp” or “W”), within DNA base pairs.

In some embodiments, a peptide may be selected from Kₙ, Rₙ, KₙWKₘ, RₙWRₘ, KₙHKₘ, RnHRₘ, KₙPKₘ, RnPRₘ, wherein n=0, 1, 2, 3, 4, 5, 6, 7, 8, 9, or 10 and wherein m=0, 1, 2, 3, 4, 5, 6, 7, 8, 9, or 10. In some embodiments, a peptide may be selected from KₘRₙ, KₙW, RₙW, KWKₙ₋₁, RWRₙ₋₁, KₙH, RₙH, KHKₙ₋₁, RHRₙ₋₁, KₙP, RₙP, KPKₙ₋₁, or RPRₙ₋₁, wherein n=1, 2, 3, 4, 5, 6, 7, 8, 9, or 10. Also included are any of these peptides which include any mixture of K and R, rather than homogenous R or homogenous K is present. Also included are any of the above peptides with more that one, for example, two, three, four, five, or six, W (also referred to herein as “tryptophan” or “Trp”), H (also referred to herein as “histidine” or “His”) and/or P (also referred to herein as “proline” or “Pro”) amino acid residues. In some embodiments, the peptide may include KWK, KWK₂, K₂WK, KWK₄, or K₄WK. Any such peptides may be DNA binding peptides.

Peptides of the present invention may also include any of the peptides described herein with an additional cysteine (also referred to herein as “Cys” or “C”) residue in the C-terminus position of the peptide, which may be utilized to accomplish the covalent linkage of the peptide to the block copolymer. Alternatively, peptides of the present invention may include an additional cysteine residue in the N-terminus position of the peptide or may include additional cysteine residues in both the N-terminus and C-terminus positions of the peptide. For example, a peptide may be selected from CKₙ, CRₙ, CKₙWKₘ, CRₙWRₘCRₙHRₘ, CRₙPKₘ, CRₙPRₘ, KₙC, RₙC, KₙWKₘC, RₙWRₘC, KₙHKₘC, RₙHRₘC, KₙPKₘC, or RₙPRₘC, wherein n=0, 1, 2, 3, 4, 5, 6, 7, 8, 9, or 10 and wherein m=0, 1, 2, 3, 4, 5, 6, 7, 8, 9, or 10. In some embodiments, a peptide may be selected from peptide includes CKₙW, CRₙW, CKWKₙ₋₁, CRWRₙ₋₁, CKₙH, CRₙH, CKHKₙ₋₁, CRHRₙ₋₁, CKₙP, CRₙP, CKPKₙ₋₁, or CRPRₙ₋₁, KₙWC, RₙWC, KWKₙ₋₁C, RWRₙ₋₁C, KₙHC, RₙHC, KHKₙ₋₁C, RHRₙ₋₁C, KₙPC, RₙPC, KPKₙ₋₁C, or RPRₙ₋₁C, wherein n=1, 2, 3, 4, 5, 6, 7, 8, 9, or 10. In some embodiments, the peptide may include CKWK, CKWK₂, CK₂WK, CKWK₄, CK₄WK, KWKC, KWK₂C, K₂WKC, KWK₄C, or K₄WKC. A peptide may be made up of amino acids that are L-isomers, D-isomers, or a mixture of L- and D-isomers.

The present invention includes any of the polymer-peptide hybrids described herein, wherein one or more nucleotide binding peptides are covalently attached to the hydrophobic block of an amphiphilic block copolymer, compositions of such polymer-peptide hybrids, methods of making such polymer-peptide hybrids and methods of using such polymer-peptide hybrids.

Synthetic vector compositions of the present invention include, as a further component, one or more isolated polynucleotides, in addition to a polymer-peptide hybrid. The isolated polynucleotides are complexed with the polymer-peptide hybrid via electrostatic interactions, and not covalent interactions, between the peptide of the polymer-peptide hybrid and the polynucleotides. Electrostatic forces include hydrogen bond forces, hydrophobic forces, van der Waals forces and salt bridges. By using small peptides to emulate the active nucleotide binding site of DNA compaction proteins and a neutral amphiphilic block copolymer as scaffold to create clustered architectural arrangements, the peptide-polymer hybrids of the present invention can combinatively self-assemble with DNA molecules into well-defined nanostructures (see FIG. 1).

As used herein, a polynucleotide contains at least two covalently linked nucleotide or nucleotide analog subunits. A nucleic acid can be a deoxyribonucleic acid (DNA), a ribonucleic acid (RNA), or an analog of DNA or RNA. Nucleotide analogs are commercially available and methods of preparing polynucleotides containing such nucleotide analogs are known

As used herein, “isolated,” with reference to a polynucleotide or other biomolecule means that the polynucleotide or other biomolecule has been separated from the genetic environment from which the polynucleotide or other biomolecule was obtained. For example, a polynucleotide naturally present in a living animal is not “isolated,” but the same polynucleotide separated from the coexisting materials of its natural state is “isolated.” Also intended as an “isolated polynucleotide” are polynucleotides that have been purified from a recombinant host cell or from a native source.

Polynucleotides include, but are not limited to, single stranded or double stranded RNA, single stranded or double stranded DNA, linearized, supercoiling, or relaxed-circular DNA, oligonucleotides, plasmids, artificial chromosomes, cDNA, cosmids, phage, or viral genomes. Polynucleotides include, but are not limited to, linear double-stranded (ds), negative-supercoiled ds, relaxed-circular ds, linear single-stranded (ss), and circular ss DNAs. Methods for the preparation of isolated polynucleotides are generally known, including standard cloning and amplification methods. A polynucleotide sequence may be any of a variety of lengths, for example, from about 20 base pairs to about 40,000 base pairs in length.

An isolated polynucleotide may be a polynucleotide sequence that encodes a polypeptide, or a biologically active fragment thereof. Such a polynucleotide sequence may be essentially any protein-encoding DNA sequence bounded by start and stop codons. Such a polynucleotide sequence may or may not include introns and may or may not include an enhancer element or a promoter located 3′ or 5′ to and controlling expression. The encoded polypeptide may be used for therapeutic applications. In some embodiments the encoded polypeptide may be an antigenic polypeptide or therapeutic protein. Such an antigenic polypeptide may be sufficient to induce a humoral or cellular immune response in an individual in which an immune response is to be elicited, and may be, for example, at least 8, at least 10, at least 12, at least 14, at least 16, at least 18, at least 20, at least 25, at least 30, at least 35, at least 40, at least 45, at least 50 amino acids in length.

An isolated polynucleotide may be an RNA interference (RNAi) sequence, that functions as a small interfering RNA (siRNA) in the inhibition of a target gene expression. Compositions of the present invention including RNAi polynucleotides may be used, for example, to treat infection by herpes simplex virus type 2 and the inhibition of viral gene expression in cancerous cells (Jiang and Milner, 2002, Oncogene; 21:6041-8), for the knockdown of host receptors and coreceptors for HIV (Crowe, 2003, AIDS; 17 Suppl 4:S103-5), in the silencing of hepatitis A (Kusov et al. 2006, J Virol; 80 (11):5599-610), and hepatitis B genes (Jia et al., 2006, Biotechnol Lett; 28:1679-85), in the silencing of influenza gene expression, the inhibition of measles viral replication (Hu et al., 2005, Acta Virol; 49:227-34), the treatment of neurodegenerative diseases, such as, for example, the polyglutamine diseases such as Huntington's disease (Raoul et al., 2006, Gene Ther 13(6):487-95), and treating cancer by silencing genes differentially upregulated in tumor cells or genes involved in cell division (Izquierdo, 2005, Cancer Gene Ther; 12:217-27 and Li et al., 2006, Cell Cycle; 5:2103-9).

Polymer-peptide hybrid and synthetic vector compositions of the present invention may be combined with other substances, including, but not limited to, for example, buffers, salts, carriers, preservatives, suspending agents, stabilizing agents, dispersing agents, and/or and particles, as appropriate. The compositions of the present invention may be formulated as pharmaceutical compositions. Such a pharmaceutical composition may include a therapeutically effective quantity of a composition including one or more polymer-peptide hybrids and one or more isolated polynucleotides according to the present invention and one or more physiologically acceptable carriers or excipients. Such pharmaceutical compositions may be formulated in any conventional manner. Such pharmaceutical compositions may be formulated for administration by any of a variety of routes, including, but not limited to, oral, buccal, parenteral, rectal, parenteral, intravenous, intradermal, transdermal, subcutaneous, topical, inhalation, or insufflation delivery, in a manner suitable for each route of administration. Formulations for parenteral administration may be administered, for example, by injection, including, but not limited to, bolus injection or continuous infusion. Formulations for injection may be presented in unit dosage form, for example, in ampoules or in multi-dose containers. Parenteral administration includes, for example, intravenous, intraperitoneal, subcutaneous, intramuscular, transepithelial, nasal, intrapulmonary, and intrathecal modes of administration. Parenteral administration may be by continuous infusion over a selected period of time.

Synthetic vector of the present invention may be formulated as a depot preparation. Such long acting formulations may be administered by implantation (for example, subcutaneously or intramuscularly) or by intramuscular injection. Synthetic vectors of the present invention may be formulated for local or topical application, such as for topical application to the skin and mucous membranes, such as in the eye, in the form of gels, creams, and lotions and for application to the eye or for intracisternal or intraspinal application. Such formulations, particularly those intended for ophthalmic use, may be formulated as 0.01%-10% isotonic solutions, pH about 5-7, with appropriate salts. The compounds may be formulated as aerosols for topical application, such as by inhalation.

The compositions of the present invention may be formulated for delivery to an individual for eliciting an immune response to a protein, or antigenic fragment thereof, encoded by an isolated polynucleotide of the composition. Such an immune response may include an antibody and/or a cellular response. The encoded antigen may be an antigen capable of eliciting an immune response against a human pathogen. Such antigen include, but are not limited to, antigens derived from HIV-1, (for example, tat, nef, gp120 or gp160, gp40, p24, gag, env, vif, vpr, vpu, rev), human herpes viruses (for example, gH, gL, gM, gB, gC, gK, gE, or gD, or derivatives thereof, or Immediate Early protein such as ICP27, ICP 47, IC P 4, ICP36 from HSV1 or HSV2), cytomegalovirus, especially human, (for example, gB or derivatives thereof), Epstein Barr virus (for example, gp350 or derivatives thereof), Varicella Zoster Virus (for example, gpl, II, III and IE63), or from a hepatitis virus such as hepatitis B virus (for example, Hepatitis B Surface antigen or Hepatitis core antigen or pol), hepatitis C virus antigen and hepatitis E virus antigen, or from other viral pathogens, such as paramyxoviruses, parainfluenza virus, measles virus, mumps virus, human papilloma viruses (for example, HPVG, 11, 16, 18, eg L1, L2, E1, E2, E3, E4, E5, E6, E7), flaviviruses (for example, Yellow Fever Virus, Dengue Virus, Tick-borne encephalitis virus, and Japanese Encephalitis Virus) or Influenza virus (such as HA, NP, NA, or M proteins, or combinations thereof), or antigens derived from bacterial pathogens such as Neisseria spp, including N. gonorrhea and N. meningitidis (for example, transferrin-binding proteins, lactoferrin binding proteins, PilC, adhesins), S. pyogenes (for example, M proteins or fragments thereof, C5A protease), S. agalactiae, S. mutans, H. ducreyi, Moraxella spp, including M. catarrhalis (for example, high and low molecular weight adhesins and invasins), Bordetella spp, including B. pertussis (for example, pertactin, pertussis toxin or derivatives thereof, filamenteous hemagglutinin, adenylate cyclase, fimbriae), B. parapertussis and B. bronchiseptica, Mycobacterium spp., including M. tuberculosis (for example, ESAT6, Antigen 85A, -B or -C, MPT 44, MPT59, MPT45, HSP10, HSP65, HSP70, HSP 75, HSP90, PPD 19 kDa [Rv3763], PPD 38 kDa [Rv0934]), M. bovis, M leprae, M. avium, M. paratuberculosis, M. smegmatis, Legionella spp, including L. pneumophila, Escherichia spp, including enterotoxic E. coli (for example colonization factors, heat-labile toxin or derivatives thereof, heat-stable toxin or derivatives thereof), enterohemorragic E. coli, enteropathogenic E. coli (for example, shiga toxin-like toxin or derivatives thereof), Vibrio spp, including V. cholera (for example, cholera toxin or derivatives thereof), Shigella spp, including S. sonnei, S. dysenteriae, S. flexnerii, Yersinia spp, including Y. enterocolitica (for example, Yop protein), Y. pestis, Y. pseudotuberculosis, Campylobacter spp, including C. jejuni (for example toxins, adhesins and invasins) and C. coli, Salmonella spp, including S. typhi, S. paratyphi, S. choleraesuis, S. enteritidis, Listeria spp., including L. monocytogenes; Helicobacter spp, including H. pylori (for example, urease, catalase, vacuolating toxin), Pseudomonas spp, including P. aeruginosa; Staphylococcus spp., including S. aureus, S. epidermidis, Enterococcus spp., including E. faecalis, E. faecium, Clostridium spp., including C. tetani (for example, tetanus toxin and derivative thereof), C. botulinum (for example, botulinum toxin and derivative thereof), C. difficile (for example, clostridium toxins A or B and derivatives thereof), Bacillus spp., including B. anthracis (for example, botulinum toxin and derivatives thereof), Corynebacterium spp., including C. diphtheriae (for example, diphtheria toxin and derivatives thereof), Borrelia spp., including B. burgdorferi (for example, OspA, OspC, DbpA, DbpB), B. garinii (for example, OspA, OspC, DbpA, DbpB), B. afzelii (for example, OspA, OspC, DbpA, DbpB), B. andersonii (for example, OspA, OspC, DbpA, DbpB), B. hermsii, Ehrlichia spp., including E. equi and, Rickettsia spp, including R. rickettsii, Chlamydia spp., including C. trachomatis (for example, MOMP, heparin-binding proteins), C. pneumoniae (for example MOMP, heparin-binding proteins), C. psittaci; Leptospira spp., including L. interrogans, Treponema spp., including T. pallidum (for example, rare outer membrane proteins), T. denticola, T hyodysenteriae, or derived from parasites such as Plasmodium spp., including P. falciparum; Toxoplasma spp., including T. gondii (for example, SAG2, SAG3, Tg34), Entamoeba spp., including E. histolytica, Babesia spp., including B. microti, Trypanosoma spp., including T. cruzi, Giardia spp., including G. lamblia, Leshmania spp., including L. major, Pneumocystis spp., including P. carinii, Trichomonas spp., including T. vaginalis, Schisostoma spp., including S. mansoni, or derived from yeast such as Candida spp., including C. albicans, Cryptococcus spp., including C. neoformans.

Synthetic vectors including polynucleotide sequences encoding antigenic polypeptides or fragments thereof, may be administered in such amount as will be prophylactically or therapeutically effective. The exact quantity may vary considerably depending on, for example, the species and weight of the mammal being immunised and the route of administration. Effective amounts of the immunological composition can be given in multiple doses, depending on the nature of the immunization regimen. For example, an initial priming dose can be given to prime the individual's immune system, and one or more subsequent doses can be given to boost the immune response generated in response to the initial priming dose. For example, the boost dose or doses can be given from 1 week to 1 year following the priming dose, and can be given periodically, for example once every 2 weeks to 6 months.

The compositions of the present invention are biocompatible. As used herein, the term “biocompatible” is meant a substance or composition that can be introduced into an subject, particularly into a human subject, without significant adverse effect. A “significant” adverse effect would be one that is considered sufficiently deleterious as to preclude introducing a substance into the subject. Moreover, for human administration, preparations should meet sterility, pyrogenicity, and general safety and purity standards as required by the FDA. Such compositions may be pyrogen-free.

The polymer-peptide hybrid and synthetic vector compositions of the present invention may also include one or more additional active agents, such as for example, a drug, a nutrient, a dye, a vitamin, a protein, a steroid, and/or an adjuvant.

The compositions of the present invention may be presented, for example, as a kit, in a pack, or as a dispenser device. Such presentations may contain one or more unit dosage forms containing the active ingredient. The kit or pack may for example, comprise metal or plastic foil, such as a blister pack. The kit, pack, or dispenser device may be accompanied by instructions for administration. The active agents may be packaged as articles of manufacture containing packaging material, an agent provided herein, and a label that indicates the disorder for which the agent is provided.

The present invention includes methods of making any of the polymer-peptide hybrids and synthetic vector compositions described herein, including, but not limited to, any of the methods described herein. As used herein, a synthetic vector composition (also referred to herein as a synthetic vector) is a composition including a polymer-peptide hybrid and one or more isolated polynucleotides.

The polymer-peptide hybrids and synthetic vectors compositions of the present invention may be used in any of a variety of applications in biotechnology, medicine, and agriculture. For example, such compositions may be used in in vitro or in vivo methods of delivering and expressing nucleotides of interest in a target cell. As used herein “in vitro” is in cell culture and “in vivo” is within the body of a subject. The present invention includes methods of transforming or transfecting host cells with a synthetic vector as described herein. As used herein, the term “host cell” includes, for example, microorganisms, yeast cells, insect cells, and mammalian cells, that can be, or have been, used as recipients for a synthetic vector. The host cell may be a prokaryotic or eukaryotic cell.

The polymer-peptide hybrids and synthetic vectors of the present invention are suitable for use in a wide variety of therapeutic applications. For example, they may be used in the expression of recombinant polypeptides, as synthetic vectors for use in gene therapy, and as vaccines. As used herein “treating” or “treatment” can include therapeutic and/or prophylactic treatments. Desirable effects of treatment include preventing occurrence or recurrence of disease, alleviation of symptoms, diminishment of any direct or indirect pathological consequences of the disease, decreasing the rate of disease progression, amelioration or palliation of the disease state, and remission or improved prognosis. The findings of the present invention can be used in methods that include, but are not limited to, methods for treating cancer, methods for treating an infection, and methods for increasing immune responses, and methods for reducing immune responses.

Synthetic vectors according to the invention which express antigenic peptides may be used as the basis of DNA vaccine compositions, immunotherapeutic compositions, methods of vaccinating, and methods of generating an immune response to an antigen. In a similar manner, vectors that encode therapeutic polypeptides may be used as the basis of therapeutic compositions, for example, therapeutic compositions for use in methods of treating cancer, infectious diseases, and chronic diseases. Cancers that may be treated include, but are not limited to, carcinoma, sarcomas, such as breast cancer, skin cancer, bone cancer, prostate cancer, bladder cancer, liver cancer, lung cancer, brain cancer, cancer of the larynx, gall bladder, pancreas, rectum, parathyroid, thyroid, adrenal, neural tissue, head and neck, colon, stomach, bronchi, kidneys, basal cell carcinoma, squamous cell carcinoma of both ulcerating and papillary type, metastatic skin carcinoma, osteosarcoma, Ewing's sarcoma, veticulum cell sarcoma, myeloma, giant cell tumor, small-cell lung tumor, gallstones, islet cell tumor, primary brain tumor, acute and chronic lymphocytic and granulocytic tumors, hairy-cell tumor, adenoma, hyperplasia, medullary carcinoma, pheochromocytoma, mucosal neurons, intestinal ganglloneuromas, hyperplastic corneal nerve tumor, marfanoid habitus tumor, Wilm's tumor, seminoma, ovarian tumor, leiomyomater tumor, cervical dysplasia and in situ carcinoma, neuroblastoma, retinoblastoma, soft tissue sarcoma, fibrosarcoma, malignant carcinoid, topical skin lesion, mycosis fungoide, rhabdomyosarcoma, Kaposi's sarcoma, osteogenic and other sarcoma, malignant hypercalcemia, renal cell tumor, polycythermia vera, adenocarcinoma, glioblastoma multiforma, leukemias, lymphomas, malignant melanomas, epidermoid carcinomas, and other carcinomas and sarcomas.

The polymer-peptide hybrids and synthetic vectors of the present invention are ideal for use in gene therapy. As used herein, “genetic therapy” involves the transfer of a heterologous polynucleotide to the certain cells, target cells, of a mammal, particularly a human, with a disorder or conditions for which therapy or diagnosis is sought. The polynucleotide is introduced into the selected target cells in a manner such that the heterologous polynucleotide is expressed and a therapeutic product encoded thereby is produced. Alternatively, the heterologous polynucleotide may in some manner mediate expression of DNA that encodes the therapeutic product, it may encode a product, such as a peptide or RNA that in some manner mediates, directly or indirectly, expression of a therapeutic product. Genetic therapy may also be used to deliver nucleic acid encoding a gene product to replace a defective gene or supplement a gene product produced by the mammal or the cell in which it is introduced. The introduced nucleic acid may encode a therapeutic compound, such as a growth factor inhibitor thereof, or a tumor necrosis factor or inhibitor thereof, such as a receptor therefor, that is not normally produced in the mammalian host or that is not produced in therapeutically effective amounts or at a therapeutically useful time. A heterologous polynucleotide encoding the therapeutic product may be modified prior to introduction into the cells of the afflicted host in order to enhance or otherwise alter the product or expression thereof.

The current invention present a versatile combinative self-assembly approach to DNA condensation and packaging by covalently attaching gene-binding oligopeptides onto an amphiphilic block copolymer scaffold, allowing for the complexation with isolated polynucleotides and the packaging of the polynucleotides inside a nanostructure. The polymer-peptide hybrid scaffold creates a clustered DNA binding effect that leads to much more efficient condensation. The polymer-peptide hybrids of the present invention may demonstrate an enhanced capability in condensing polynucleotides into stable, compact structures, including, but not limited to toroids, rods, and spheres. Such condensate may demonstrate, for example, enhanced DNA stabilization and protection against double strand breakage, thermal degradation, and/or nuclease degradation. Such condensates are well suited for use as synthetic vectors for the packaging and delivery of polynucleotides in gene therapy applications.

With the present invention, the hydrophilic block of the amphiphilic block copolymer may be further conjugated with a wide variety of functional groups. Such functional groups may facilitate, for example, specific cell targeting, extracellular transport, endosomal release, receptor-mediated cellular uptake, endocytosis, nuclear transport, transnuclear localization, transfection, and/or gene regulation.

Compositions of the present invention may be administered to a subject. As used herein, the term “subject” includes, but is not limited to, humans, non-human vertebrates, plants, and invertebrates. Non-human vertebrates include livestock animals, companion animals, and laboratory animals. Non-human subjects also include non-human primates as well as rodents, such as, but not limited to, a rat or a mouse. Non-human subjects also include, without limitation, chickens, other birds, horses, cows, pigs, goats, sheep, dogs, cats, guinea pigs, hamsters, mink, and rabbits. As used herein, the terms “subject,” “individual,” “patient,” and “host” are used interchangeably. In preferred embodiments, a subject is a mammal, particularly a human.

The present invention is illustrated by the following examples. It is to be understood that the particular examples, materials, amounts, and procedures are to be interpreted broadly in accordance with the scope and spirit of the invention as set forth herein. For any method disclosed herein that includes discrete steps, the steps may be conducted in any feasible order. And, as appropriate, any combination of two or more steps may be conducted simultaneously.

### EXAMPLES

### Example 1

### Grafting Short Peptides onto Polybutadieneblock-Poly(Ethylene Oxide): A Platform for Self-Assembling Hybrid Amphiphiles

The peptide in peptide-containing hybrid amphiphiles usually plays the role of a hydrated hydrophilic head group rather than that of a hydrophobic tail (Hartgerink et al., 2001, Science; 294:1684-1688; Schlaad and Antonietti, 2003, Eur Phys J E; 10:17-23; and Schlaad, 2006, Adv Polym Sci; 202:53-74). However, chiral peptide interactions within a dense hydrophobic core of an amphiphile assembly might better mimic the hydrophobic interactions that drive the collapse and ordering in protein folding and highly specific protein-protein associations (Miao et al., 2004, J Mol Biol; 344:797-811). The goal of this example was to make self-assembling hybrid amphiphiles that integrate peptides within the hydrophobic core. More-practical reasons for attaching short peptides onto synthetic polymers involve biomedical applications, including, but not limited to, amino acids chosen from those often found in protein cores (cysteine, phenylalanine, tryptophane, etc.) can help to tune the solubility of hydrophobic drugs, and short peptides with less than six repeat units are less antigenic than long polypeptides or proteins. See, for example, Rudensky et al., 1991, Nature; 353:622-627; Yewdell and Bennink, 2001, Curr Opin Immunol; 13:13; Deming, 2002, Adv Drug Delivery Rev; 54:1145-1155; and Velonia et al., 2002, J Am Chem Soc; 124:4224-4225.

Purely synthetic block copolymer amphiphiles can self assemble into various mesostructures (e.g. spherical micelles, wormlike micelles, and vesicles) in aqueous solutions (Jain and Bates, 2003, Science; 300:460-464; Firster et al., 1996, Chem Phys; 104:9956-9970; and Discher and Eisenberg, 2002, Science; 297:967-973) by similar principles to the hydrophobic effect operative for surfactants (i.e. minimization of the contact of the hydrophobic blocks with water) (J. N. Israelachvili, Intermolecular and Surface Forces, Academic Press, London, 1985; and P. Alexandridis, B. Lindman, Amphiphilic Block Copolymers:Self-assembly and Applications, Elsevier, New York, 2000). Morphological phase diagrams mapped out for nonionic block copolymers of polybutadiene-block-poly(ethylene oxide) (PBD-b-PEO) show that their self-assembly structures in water are largely determined by the weight fraction of the hydrophilic PEO block (w_{EO}) and that decreasing w_{EO} disfavors spherical micelles and favors structures with lower mean curvature such as wormlike micelles and vesicles (Jain and Bates, 2003, Science; 300:460-464). Although the structures of synthetic block copolymers can be tuned, the hybridization of synthetic copolymers with peptides leads to a further level of sophistication and functionality of the self-assembled structures (Hartgerink et al., 2001, Science; 294:1684-1688; and Schlaad and Antonietti, 2003, Eur Phys J E; 10:17-23). Helical superstructures have recently been prepared from peptide hybrid amphiphiles, typically using long polypeptides as the hydrophilic block, such as polystyrene-block-poly(isocyanodipeptide) (Cornelissen et al., 1998, Science; 280:1427-1430) and polybutadiene-block-poly(1-glutamate) (Kukula et al., 2002, J Am Chem Soc; 124:1658-1663; and Chkcot et al., 2002, Angew Chem; 114:1395-1399; Angew Chem Int Ed; 41:1339-1343). These peptide hybrids were generally synthesized by advanced techniques such as metal-catalyzed polymerization of isocyanopeptides or ringopening polymerization of the α-amino acid N-carboxyanhydride.

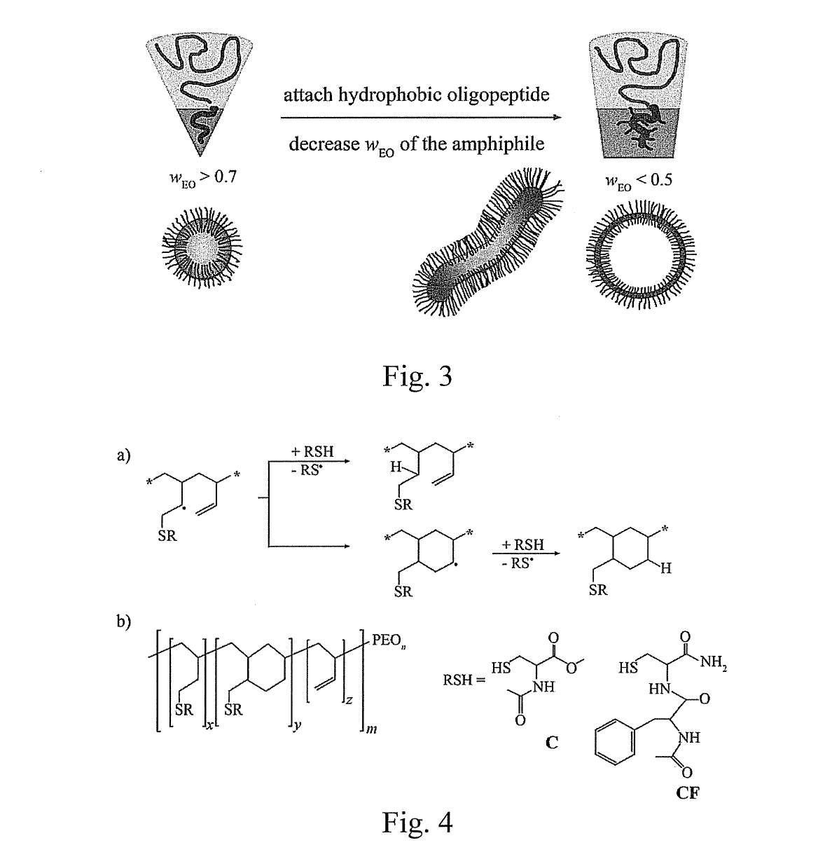

This example introduces a new type of self-assembling peptide hybrid amphiphiles by using synthetic PBD-b-PEO as the backbone and grafting cysteine-containing oligopeptides onto the hydrophobic PBD segment through a free radical addition reaction. Modification with difluorocarbene was used in the solid-state to tune the self-assembly behavior of block copolymers (Ren et al., 2000, Macromolecules; 33:866-876); in aqueous solutions, attaching short hydrophobic peptides decreases the w_{EO}, value of the amphiphile and thus shifts the preferred self-assembly morphology towards those with lower curvatures, namely, from spherical micelles to wormlike micelles and vesicles (FIG. 3). Initial studies also suggest that chiral peptide interactions within the hydrophobic core foster the formation of helical super structures.

Two PBD-b-PEO copolymer samples with similar number-average molecular weight (Mₙ) but different weight fractions of PEO were used: PBD₁₄-b-PEO₉₃ (1, Mₙ=4900 gmol⁻¹, W_{EO}=0.83) and PBD₂₅-b-PEO₇₅ (2, Mₙ=4700, w_{EO}=0.70). As reported earlier, PBD-b-PEO can be functionalized through free-radical addition of ω-functional mercaptans (RSH) without altering the molecular-weight distribution (MWD) (Justynska et al., 2005, Polymer; 46:12057-12064; and Justynska et al., 2004, Macromol Rapid Commun; 25:1478-1481). The number of functional groups attached to the PBD chain is usually lower than the number of converted double bonds owing to a side reaction of the radical species, namely, the formation of six-membered cyclic units (FIG. 4A). The radical addition route was extended from mercaptans to chiral cysteine-containing oligopeptides. The route was first tested on an L-cysteine derivative (c) and then on the dipeptide (L,L)-cysteine-phenylalanine (CF) as model oligopeptide to prove the applicability of this route (FIG. 4B).

Addition reactions of C onto 1 and 2 were performed in refluxing tetrahydrofuran (THF) using azoisobutyronitrile (AIBN) as the radical source ([C═C]₀/[C₀]/[AIBN]₀=1:10:0.33). Conversion of PBD double bonds came to completion within 24 hours and products 1-C and 2-C exhibited the same narrow MWDs as the precursors (PDI≦1.05 (Justynska et al., 2005, Polymer; 46:12057-12064). In the case of CF, the molar ratio was chosen to be [C═C]₀/[CF]₀/[AIBN]=1:2.5:0.33 for economic reasons. Further decreasing the amount of CF bears the risk that the PBD chains will undergo intermolecular cross-linking. Because of the poor solubility of CF in THF, the radical addition reaction was carried out in N-methylpyrrolidone (NMP) as the solvent. The ¹H NMR spectra (1-CF shown in FIG. 5A, 2-CF, discussed in more detail below) shows signals of the dipeptide at δ=4.3, 4.5 (α-CH), 7.1-7.3 (phenyl), and 8.1 ppm (NH); the signals of the thioether linkage —CH₂SCH₂— arise at δ≈2.7 and 2.9 ppm. Resonances at δ=4.8-5.6 ppm indicate the presence of residual double bonds. Quantitative analysis of signal intensities relative to that of PEO (δ≈3.6 ppm) suggested that both samples contain about 10 CF units and 2-3 unreacted butadiene units. SEC (with differential refractive index (RI) and UV detectors) of 1-CF (FIG. 5B) confirmed the attachment of CF units onto the polymer (Uvabsorption at 270 nm) and the preservation of the narrow MWD (polydispersity index <1.2).

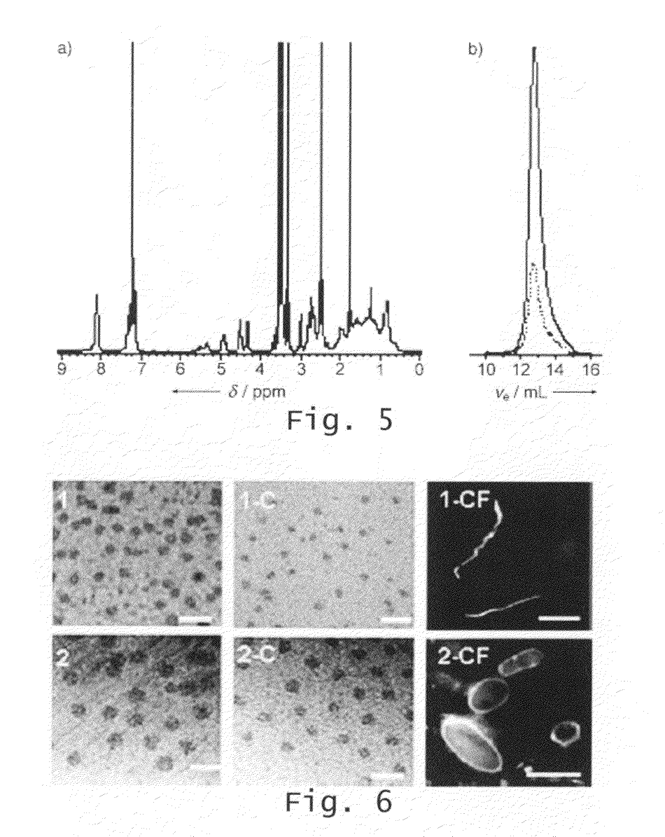

As mentioned above, the morphology of the PBD-b-PEO self-assembled structures in water is dictated by the w_{EO} value. Spherical micelles predominate when w_{EO}>0.55, whereas wormlike micelles form when w_{EO}≈0.50-0.55 and vesicles form when w_{EO} is well below 0.5 (Jain and Bates, 2003, Science; 300:460-464). The precursor PBD-b-PEO samples 1 (w_{EO}=0.83) and 2 (w_{EO}=0.70) form spherical micelles at 0.1% w/w in water, as visualized by cryogenic transmission electron microscopy (cryo-TEM; FIG. 6). The dark spots observed in the micrographs correspond to the hydrophobic cores of the micelles, which are about 12 and 20 nm in diameter for 1 and 2, respectively. The hydrodynamic diameters of the micelles determined by dynamic light scattering (DLS) are 27 and 45 nm, respectively.

Grafting C onto 1 and 2 had no effect on the shape of micelle cores, yet decreased their size by 10-30% (FIG. 6). The hydrodynamic diameters of the micelles also decreased to about one third that of the precursor micelles. The formation of smaller-sized micelles can be attributed to the slight hydrophilic nature of cysteine, which increases the overall hydrophilic fraction of the hybrids and the corresponding interfacial curvature.

In contrast, dipeptide CF is hydrophobic, and grafting CF onto 1 and 2 leads to a decrease of w_{EO} and shifts the morphology towards those with smaller curvatures, that is, wormlike micelles and vesicles. Fluorescence microscopy (FM) revealed giant wormlike micelles with contour lengths of 10-15 μm for 1-CF (w_{EO}=0.54) and vesicles with diameters of 2-5 μm for 2-CF (w_{EO}=0.43) (FIG. 6). FM is widely used to study cellular structures and dynamics, but has recently been established also as a reliable and convenient technique for visualizing micrometer-sized wormlike micelles and vesicles (Discher et al., 1999, Science; 284:1143-1146; Dalhaimer et al, 2004, J Polym Sci. Part B; 42:168-176; and Geng and Discher, 2005, J Am Chem Soc; 127:12780-12781). Unlike cryo-TEM, FM allows direct observation of samples in aqueous solutions without requiring fixation and constraint in thin films, and thus provides more equilibrated and convenient access to length and dynamic measurements of soft objects. The w_{EO} values of the CF-grafted PBD-b-PEO that correspond to wormlike micelles and vesicles are quantitatively comparable to the PBD-b-PEO morphological phase diagram (Jain and Bates, 2003, Science; 300:460-464), thus suggesting that the self-assembly of such hybrids in water is likewise dictated by the hydrophobic-hydrophilic balances.

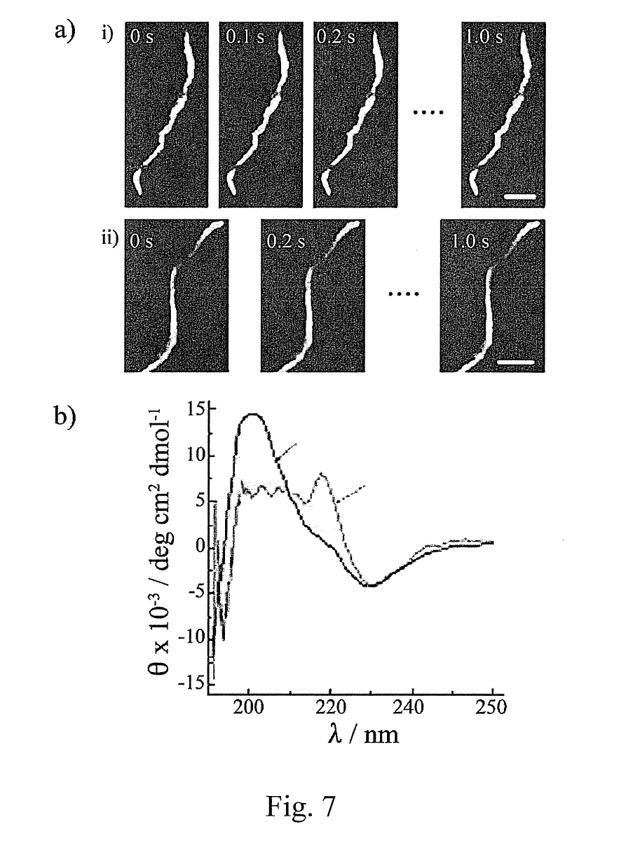

Integrating the dipeptide CF into PBD-b-PEO not only changed the hydrophobic-hydrophilic ratio of the amphiphile, but also introduced hydrogen-bonding, π-π, and chiral interactions into the hydrophobic core of the assembly, which might induce the formation of helices. Helices often emerge in nature from such interactions, for example, in proteins and DNA. As helices are generally more observable in fibrous structures, the giant 1-CF wormlike micelles was examined in more detail. FM revealed the existence of both right- and left-handed defected helical structures with supermolecular pitches of a few micrometers (FIG. 7A). Sequential snapshots of individual 1-CF wormlike micelles confirm that their helical curvature is rigid, whereas pristine PBD-b-PEO wormlike micelles are extremely soft and flexible and exhibit large thermal fluctuations (Dalhaimer et al., 2003, Macromolecules; 36:6873-6877). Analysis of the circular dichroism (CD) spectrum of an aqueous solution of 1-CF wormlike micelles (FIG. 7B) indicates a near racemic mixture of helices with right-handed and left-handed screw senses (see Materials and Methods, discussed below). Although investigations continue, the capability of such amphiphilic hybrids that contain hydrophobic peptides to self-assemble into helical superstructures is evident.

In conclusion, this example presents a new type of peptide hybrid amphiphiles by directly grafting cysteine-containing oligopeptides onto synthetic PBD-b-PEO copolymers. The morphologies (spherical micelles, wormlike micelles, and vesicles) of such self-assembled hybrid amphiphiles may be tuned by controlling the hydrophobic-hydrophilic ratios. Helical superstructures also arise from the chiral peptide interactions inside the assembly core. This new class of hybrids allows the integration of functionalities into the assembly core for various applications. Future investigations will likely include exploring the attachment of different functional oligopeptides, studying their self-assembly and superstructures, and pursuing their biomedical applications.

### Materials and Methods

N-Acetyl L-Cysteine methyl ester (c) and all other chemicals were purchased as high-purity reagent-grade materials from Sigma-Aldrich and were used as received. Model dipeptide (L,L)-cysteine-phenylalanine (CF) (N-terminal: acetylation, C-terminal: amidation, purity: >90%) was ordered from Genscript Corporation. Tetrahydrofuran (THF) and N-methyl-2-pyrrolidone (NMP) were degassed and purified before use. The block copolymer precursors (1: PBD₁₄-b-PEO₉₃ and 2: PBD₂₅-b-PEO₇₅; subscripts denote the average number of repeating units) were prepared by sequential anionic polymerization of buta-1,3-diene and ethylene oxide according to a procedure published elsewhere (Chkcot et al., 2002, Angew Chem; 114:1395-1399; Angew Chem Int Ed; 41:1339-1343; and Ren et al., 2000, Macromolecules; 33:866-876). Samples exhibited a narrow molecular-weight distribution (polydispersity index, PDI=1.02 and 1.05, for 1 and 2 respectively) and did not contain any homopolymer impurities, as revealed by size-exclusion chromatography (SEC). Analysis of the microstructure by ¹H NMR indicated that the PBD consisted of about 95% 1,2-units.

Radical addition of C and CF onto PBD-b-PEO. The reaction flask containing polymer (1 or 2), peptide (c) or CF), and 2,2-azoisobutyronitrile (AIBN) was degassed for 15 minutes and then dry solvent (C: THF, CF: NMP) was added. The resulting ˜3 wt % solution was heated to about 70° C. and stirred for 24-48 hours under an argon atmosphere. The solvent was removed under vacuum, and the crude product was re-dissolved in water and dialyzed against bi-distilled water to remove unreacted mercaptan. After freeze-drying, the final product was collected as a white-light yellow, fluffy material in a 55-75% yield.

Characterization of polymers. ¹H NMR was performed on a Bruker DPX-400 spectrometer operating at 400.1 MHZ; the solvents used were CDCl₃ and DMSO-d₆. SEC with simultaneous UV and RI detection was performed (I) in THF (flow rate: 1.0 mL/min) at 25° C. using a column set of three 300×8 mm MZ-SDplus (spherical PS particles with an average diameter of 5 μm) columns with pore sizes of 10³, 10⁵, 10⁶ Å (1-C and 2-C) and (ii) in N-methyl-2-pyrrolidone (NMP+0.5 wt % LiBr; flow rate: 0.5 mL/min) at 70° C. using a column set consisting of two 300×8 mm columns filled with 5 μm PS particles, PL-gel (10² Å) and PSS-gel (10³ Å) (1-CF and 2-CF). Calibration was done with PS standards (THF).

Preparation and characterization of peptide hybrid assemblies in water. The peptide amphiphile hybrids were directly dissolved in water (concentration: 0.1 wt %), and solutions were equilibrated at 60° C. for overnight. Olympus IX71 inverted fluorescence microscope with a 60× objective and a Cascade CCD camera was used to visualize peptide hybrid worm micelles and vesicles. A hydrophobic fluorophore dye (PKH 26) was added to the OCL worm micellar aqueous solutions, and 2 μL sample was used in the chamber formed between glass slide and cover slip and approximately 20 pictures were taken per sample. Spherical micelles of PBD-PEO and cysteine grafted polymers were imaged with a JEOL 1210 Cryogenic transmission electron microscope (cyro-TEM). Sample preparations and procedures were published elsewhere (Kukula et al., 2002, J Am Chem Soc; 124:1658-1663). Dynamic light scattering (DLS) was performed at 25° C. with an ALV goniometer and an ALV-5000 digital correlator (ALV GmbH, Langen, Germany) with a He—Ne laser (intensity: 34 mW, λ=633 nm) as the light source. DLS auto correlation functions were measured at different scattering angles and evaluated with CONTIN. Hydrodynamic diameters of aggregates were calculated by applying the Stokes-Einstein equation. Circular Dichroism analysis was performed at room temperature with a JASCO J 715 spectrometer using quartz cells with 0.5 mm optical path length.

¹H NMR on 2-C shows the disappearance of the double bond proton signals, indicating that the conversion of PBD double bonds came to completion. Furthermore, it was found by the integration of characteristic NMR signals (cysteine α-CH at δ=4.8 ppm, PEO: —OCH₂— at δ=3.4-3.7 ppm, newly formed thioether linkage —CH₂SCH₂— at d=2.7 and 2.9 ppm) that about 18 C units were bound to the PBD chain. SEC shows that the molecular weight distribution of 2-C is as narrow as that of the precursor 2 (PDI=1.05). The CD spectrum of 1-CF worm micelle in aqueous solution shows different signals from the dipeptide CF (saturated aqueous solution) at the wavelength 180-230 nm, suggesting the possible formation of secondary structures in 1-CF worm micelle solutions. CDProsoftware (CDSSTR) was used to deconvolute the CD data of 1-CF for determining the secondary structure fractions. This program implements the variable selection method using a reference set and assigns the best-fit secondary structure (input: molar ellipticity within wavelength 180-240 nm). CDSSTR analysis on 1-CF worm micelle aqueous solution shows that the fractions of right-handed helix and left-handed helix are 0.19 and 0.22 respectively.

Example 1 can also be found as Geng et al., “Grafting Short Peptides onto Polybutadieneblock-poly(ethylene oxide): A Platform for Self-Assembling Hybrid Amphiphiles,” 2006, Angewandte Chemie 45(45):7578-81 and its online Supporting Information (available on the world wide web at wiley-vch.de/contents/jc_{—}2002/2006/z602739_s.pdf), each of which are hereby incorporated by reference in their entirety.

### Example 2

### DNA Packaging Via Combinative Self-Assembly

With this example, a novel and versatile DNA packaging approach was developed by grafting DNA-binding oligopeptides onto a polymer scaffold to combinatively self-assemble with DNA into compact nanostructures (FIG. 1). This packaging approach can be used in the development of synthetic gene vectors, as well as to elucidate the gene packaging mechanisms in general. The polymer scaffold creates a spatial clustering arrangement and multiplies the oligopeptide binding sites with DNA. The DNA condensation studies presented in this example demonstrate that the clustering of oligopeptides results in more effective DNA compaction than free oligopeptides and demonstrate that the clustering density has a profound influence on DNA packaging.

In this example, oligopeptides were grafted onto the hydrophobic segment polybutadiene (also referred to herein as “PBD”) of the amphiphilic block copolymers of PEG-b-PBD. The gene-binding oligopeptide grafted PBD segment is expected to complex with DNA to form the core, and the PEG segment to form a protecting corona.

For grafting, the modular free radical addition route described in Example 1 (see also Geng et al, (Geng et al. 2006, Angew Chem; 45:7478-7581) was used. By using cysteine as the terminal linker of the oligopeptide sequence, the thiol group of the cysteine reacts with the double bonds of PBD by the free radical addition reaction and “clicks” the desired oligopeptide sequence onto PEG-b-PBD (FIG. 2). In this example, a simple gene-binding tripeptide sequence, lysine-tryptophan-lysine (KWK), was synthesized by solid phase peptide synthesis (SPPS) and used for grafting. It is known that KWK binds with DNA via two kinds of interactions: electrostatic interactions between the positively charged lysine residues and negatively charged phosphate DNA backbone, and the intercalation of aromatic tryptophan within DNA base pairs (Sparrow et al., 1998, Adv Drug Delivery Rev; 30:115-131). PEG₇₅-b-PBD₂₅ with the weight fraction of PEO, w_{EO}˜0.7, was used as the polymer scaffold. The free radical grafting has led to well defined molecular architectures, sustaining the narrow molecular weight distribution of PEG-b-PBD precursor, synthesized by living anionic polymerization (polydispersity index 1.05). The grafting density can be controlled and tuned by changing the molar ratio between the thiol group and the PBD double bonds.

In this example, two combinative polymer-peptide hybrids with different grafting density were synthesized; PEG₇₅-b-PBD₂₅ with four peptide grafts, was designated as “PP4,” and PEG₇₅-b-PBD₂₅ with eight peptide grafts, was designated as “PP8.” The grafting procedure, and characterization of PP4 and PP8 by NMR and gel permeation chromatography are included in the Materials and Methods section, below.

### Materials and Methods

All chemicals were purchased from Sigma-Aldrich. KWK and CKWK were synthesized using standard Fmoc SPPS procedure with HOBT, HBTU, and DIPEA couplings, followed by N-capping with acetic anhydride. Peptides were analyzed by ESI-MS and ¹H NMR before Grafting. Amphiphilic block copolymer of PEG-b-PBD was synthesized by the well-established living anionic polymerization. The structure of PEG-b-PBD was confirmed by NMR and its polydispersity (PDI) was determined by Gel Permeation Chromatography to be 1.05.

λ-phage DNA 250 μg/mL stored in 10 mM Tris buffer/0.5 mM EDTA was purchased from New England Biolabs. This DNA stock solution was dialyzed against 10 mM sodium cocadylate buffer (pH 6.5) containing 0.5 mM EDTA and further diluted to 100 μg/mL before use.

Grafting of Cysteine containing peptide to PEG-b-PBD. CKWK was grafted to PBD₂₅-PEO₇₅ according to procedure of Example 1 (see also Geng et al. 2006, Angew Chem.; 45:7478-7581). Briefly, The reaction flask containing polymer (PBD₂₅-PEO₇₅), peptide (CKWK), and 2,2 azoisobutyronitrile (AIBN) was degassed for 30 minutes and then dry solvent, 1-methyl-2-pyrrolidinone (NMP), was added. Different molar ratios between [C═C]₀ and [—SH]₀ were used in order to achieve different grafting density. [C═C]₀/[—SH]₀/[AIBN]₀=1:3:0.33 and 1:5:0.33 were used for PP4 and PP8 synthesis respectively. The resulting solution was heated to 70° C. and stirred for 48 hours under an argon atmosphere. AIBN was reinjected after 24 hours. After the reaction was complete, NMP was removed under vacuum. The crude product was re-dissolved in water and dialyzed against pure water to remove the unreacted peptides. The product was freeze-dried and collected for ¹H NMR and Gel Permeation Chromatography (GPC) analysis.

In ¹H NMR analysis on PP4 and PP8, the characteristic signals of the grafted oligopeptide were observed at δ=6.6-7.6 (tryptophan), and 8.1 ppm (NH); the signal of the thioether linkage —CH₂SCH₂— arise at δ˜2.7 and 2.9 ppm. Resonances at δ=4.8-5.6 ppm indicate that the conversion of PBD double bonds did not come to completion. The quantitative analysis of signal intensities relative to that of PEG at δ˜3.6 ppm reveals that PP4 chain contains about 4 KWK units and 21 unreacted butadiene units, whereas PP8 contains 8 KWK units and 17 unreacted butadiene units. GPC analysis showed single narrow peak for PP4 and PP8 respectively, indicating the narrow polydispersity of the PBD-b-PEG scaffold has been preserved during the grafting process.

Preparation of DNA complexes. DNA-KWK, DNA-PP4, DNA-PP8 complexes were prepared by simply mixing of an equal volume of 100 μg/mL DNA and KWK, PP4, PP8 with desired concentrations in the 10 mM sodium cocadylate buffer. The mixture solution was vortexed for 30 seconds and allowed to equilibrate at room temperature for a few hours. The final DNA concentration was set at 50 μg/ml. For comparison purpose, same final stoichiometric KWK concentration at 64 μM was used for the three complex systems: 64 μm free KWK, 16 μM PP4 that contains 16×4=64 μM KWK, and 8 μM PP8 that contains 8×8=64 μm KWK.

Characterization of DNA complexes structure by Atomic Force Microscopy. DNA complexes were deposited on freshly cleaved mica and then allowed to air dry. Tapping mode AFM imaging was performed on a Digital Instruments Nanoscope Ma scanning probe microscope with a multimode head. Silicon probes (VistaProbes T300) with spring constant 40 N/m, resonant frequency, 300 kHz was used to obtain all images.

DNA Melting Studies. DNA melting studies on native DNA and DNA complexes in 10 mM sodium cocadylate buffer were performed on a Carey 100 UV-Vis. DNA absorbance at 260 nm was monitored with temperature, slowly increasing from 50° C. to 95° C. at 1° C./min heating rate. For DNA-KWK, DNA-PP4, DNA-PP8 complexes, weak background absorbances from KWK, PP4 and PP8 were directly subtracted from the measurements, by using the corresponding KWK, PP4 and PP8 in 10 mM sodium cocadylate buffer as reference cells.

### Results and Discussion

Long linear lambda phage DNA, λ-DNA, (double-stranded, 48 kbp, MW=3×10⁷ Da, contour length—17 μm) in dilute aqueous solution (100 μg ml⁻¹ DNA in 10 mM pH=6.5 sodium cacodylate buffer containing 0.5 mM EDTA) was used for packaging studies. Complexes between DNA and each of KWK, PP4, and PP8 were prepared by simply mixing an equal volume of both components in the same buffer at room temperature. The final DNA concentration was set at 50 μg ml⁻¹ throughout the studies in this example. The DNA-KWK, DNA-PP4 and DNA-PP8 complex structures were investigated by atomic force microscope (“AFM”) on mica substrates. AFM is a well established, reliable technique for studying DNA condensate structures, and it has been proved that absorbance of DNA condensates onto mica does not significantly change their structures.

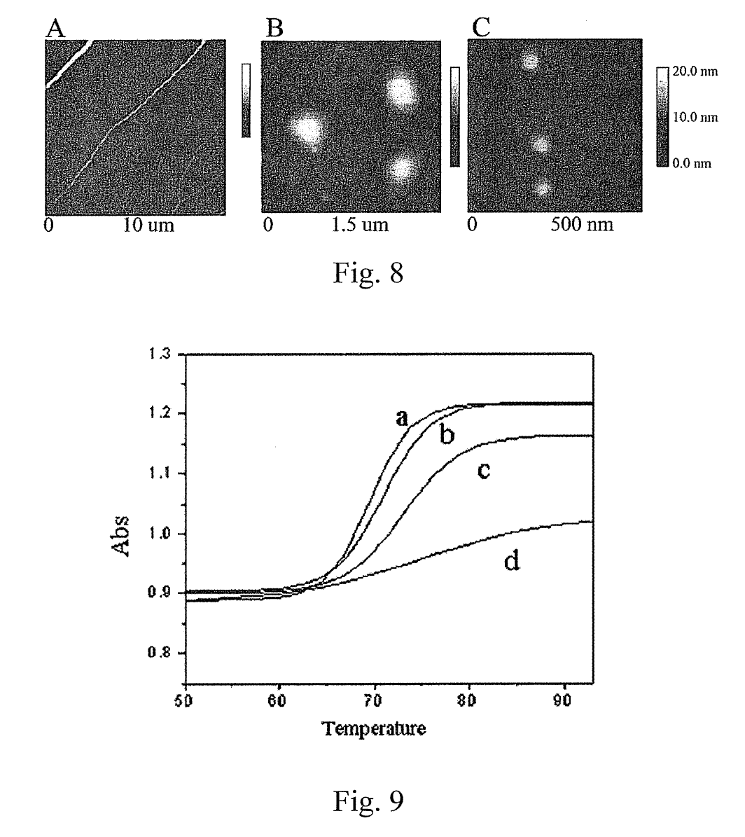

The free gene-binding tripeptide KWK is unable to induce λ-DNA compaction, even over a wide peptide concentration range. A typical DNA-KWK complex structure at 64 μM KWK visualized by AFM is shown in FIG. 8A. No DNA compaction was observed. Instead, long extended thick bundles of DNA were found. The width of each bundle was measured to be between 5 to 10 nm. Considering an individual double stranded-DNA is about 2 nm in width, the much thicker DNA bundles observed here indicate that each bundle was aggregated by at least two ds-λDNA. Upon careful examination of some DNA bundles, single ds-λDNA (˜2 nm width) can be seen to protrude from bundle stems (FIG. 8A). Since KWK has only two lysines, with one at each end, it seems that this gemini-like dication tends to bridge DNA intermolecularly rather than inducing intramolecular compaction. This is consistent with Manning's electrolyte counterion theory that a counterion with higher than 3 valency is needed to induce DNA compaction (Manning, 1978, Q Rev Biophys; 11:179-246).

Grafting KWK onto the PEG-b-PBD polymer scaffold dramatically changed its complexation behavior with DNA. Control experiments showed negligible interactions between DNA and the neutral amphiphilic PEG-b-PBD backbone with the linker cysteine (where —SH is already converted into a thiol ether group) in dilute solutions. Therefore, for the polymer-KWK hybrids, PP4 and PP8, their major interactions with DNA also originate from KWK. However, under the same stoichiometric KWK concentration at 64 mM, i.e. 16 μM PP4 and 8 μM PP8, respectively, the polymer-KWK hybrids PP4 and PP8 are able to self-assemble with λ-DNA and condense the long DNA into compact structures (FIGS. 8B and 8C). For PP4 with a lower density of KWK grafts, partial DNA compaction was observed by AFM (FIG. 8B). Surrounding the compacted portion with an average size ˜300 nm in diameter, a portion of uncompacted DNA molecule is visible in the periphery (FIG. 8B). In comparison, PP8 with twice as high grafting density is able to completely compact λ-DNA into much smaller nanostructures (FIG. 8C).

Statistical analysis demonstrated that such DNA-PP8 complexes are all disk-like in shape and have rather narrow size distribution with an average diameter around 100 nm and height around 10 nm. This significant difference is probably due to that by clustering the oligopeptides, they can bind, bend and/or distort DNA in a more cooperative fashion into compact structures. With higher grafting density, the number of the oligopeptides being clustered by the polymer scaffold increases and they are brought to closer proximity, leading to more efficient DNA compaction. The disk-like DNA complex structure formation, instead of the more ordered toroids, may relate to the nature of KWK, which contains only two lysine residues separated by a bulky tryptophan spacer. The size of the PBD polymer scaffold may also exert some influence on the DNA complex formation. By using different gene-binding oligopeptide sequences and a PBD-b-PEG scaffold with different degrees of polymerization (i.e. molecular weight), different DNA complex structures can potentially be formed and tuned.

The stability of DNA-KWK, DNA-PP4 and DNA-PP8 complexes was investigated by DNA melting studies. Breakage of double-stranded DNA into single strands was monitored by an increase in the DNA absorbance at 260 nm, due to the disruption of hydrogen bonds between base pairs (i.e. hyperchromic effect). For native λ-DNA, Tₘ, the temperature at which 50% of ds-DNA dissociates, was determined to be 70° C. (FIG. 9, curve a). For melting studies on DNA-KWK, DNA-PP4 and DNA-PP8 complexes, weak background absorbances from the corresponding KWK, PP4, PP8 at 260 nm have been subtracted to achieve accurate DNA absorbance measurements. B_{y} complexing with 64 μM KWK, Tₘ was only slightly shifted, showing no strong intramolecular stabilization on the double-stranded λI-DNA (FIG. 9, curve b). This is consistent with the AFM results, where only intermolecular DNA bridging by KWK but no DNA compaction was observed. In comparison, the DNA-PP4 complex where DNA was partially compacted showed a notable Tₘ shift to 75° C. (FIG. 9, curve c). For the completely compacted DNA-PP8 complex, a tremendously improved stability was shown. A much slower ds-DNA dissociation curve was observed (FIG. 9, curve d). Even by the upper limit of DNA melting studies in aqueous solutions 95° C., that is, right below the boiling point of water, only a slight change in DNA absorbance was detected, suggesting that the majority of double stranded DNA remained intact.

In conclusion, this example presents a novel and versatile combinative self-assembly approach for the packaging of genetic materials into stable, compact nanostructures. This approach will further our understanding of how the spatial arrangement of gene-binding oligopeptides affects gene packaging, and serves as a promising new designing platform for synthetic gene vector development. Further experiments will utilize different gene-binding oligopeptide sequences, molecular architecture of the polymer scaffold, and solution conditions (buffer, salt, pH) in order to gain further understanding and control over the combinative self assembly behavior with genes.

Example 2 has also published as “DNA packaging via combinative self-assembly,” Haley et al., 2008, Mol. BioSyst; 4:515-517 (DOI: 10.1039/b800220g) and its accompanying online supporting information (available on the worldwide web at.rsc.org/suppdata/MB/b8/b800220g/b800220g.pdf), each of which are hereby incorporated by reference herein, in their entirety.

### Example 3

### Effect of Clustered Binding on DNA Condensation

DNA condensation in-vitro has been studied as a model system to reveal common principles underlying gene packaging in biology, and as the critical first step towards the development of non-viral gene delivery vectors. In this study, a bio-inspired approach was used, where small DNA binding peptides are controllably clustered by an amphiphilic block copolymer scaffold, to reveal the effect of clustered peptide binding on the energetics, size, shape and physical properties of DNA condensation in-vitro. This provides insights into the general architectural effect of gene binding proteins on the DNA condensation process. Moreover, the versatility afforded by regulating the clustering density and composition of peptides may provide a novel design platform for gene delivery applications in the future.

Condensation of long strands of DNA into compact, ordered structures is an important biological process for gene protection, storage and replication, and has attracted tremendous interest to a broad spectrum of scientific communities. DNA condensation in-vitro has been pursued as a model system to study the phase transition phenomena of polyelectrolytes (Manning, 1978, Q Rev Biophys; 11:103-178), to reveal common principles underlying gene packaging in biology (Klimenko et al., 1967, J Mol Biol; 23:523-533; Cerritelli et al., 1997, Cell; 91:271-280) and as the critical first step towards the development of non-viral gene delivery vectors (Luo and Saltzman, 2000, Nat Biotechnol; 18:33-37; Mintzer and Simanek, 2009, Chem Rev; 109:259-302). Historically, toroids, where loops of DNA double helix pack in hexagonal arrays, have attracted the most attention and are considered as the predominant in-vitro DNA condensate structure (Gosule and Schellman, 1976, Nature; 259:333-335; Widom and Baldwin, 1980, J Mol Biol; 144:431-453; Conwell et al., 2003, PNAS; 100:9296-9301; and Hud et al., 1995, PNAS; 92:3581-3585). Occasionally, metastable rod-like DNA condensates have also been discovered and are attracting increasing interest in recent years (Arscott et al., 1995, Biopolymers; 36:345-364; and Vilfan et al., 2006, Biochemistry; 45:8174-8183).

A wide variety of materials have been explored as DNA condensing agents, ranging from the original small natural amines ((for example, spermidine and spermine) (Gosule and Schellman, 1976, Nature; 259:333-335)) and multivalent cations (for example, Co(NH₃)₆³⁺) (Widom and Baldwin, 1980, J Mol Biol; 144:431-453)) to much more complex materials, such as lipids (Radler et al., 1997, Science; 275:810), crowding agents (Laemmli, 1975, PNAS; 72:4288-4292), dendrimers (Haensler and Szoka, 1993, Bioconjugate Chem; 4:372-379), peptides (Sparrow et al., 1998, Adv Drug Delivery Rev; 30:115-131; and Wadhwa et al., 1997, Bioconjugate Chem; 8:81-88) polyamines, (Wagner et al., 1990, PNAS; 87:3410-3414; Choi et al., 1998, Bioconjugate Chem; 9:708-718; Mislick et al., 1995, Bioconjugate Chem; 6:512-515; and Zauner et al., 1998, Adv Drug Delivery Rev; 30:97-113) and their corresponding block copolymers (Kataoka et al., 1996, Macromolecules; 29:8556-8557; and Kakizawa and Kataoka, 2002, Adv Drug Delivery Rev; 54:203-222). For small natural amines and multivalent cations, the mechanism and pathway of their DNA condensation have been vigorously studied and are fairly well understood, providing invaluable foundation for later studies (Conwell et al., 2003, PNAS; 100:9296-9301; and Bloomfield, 1997, Biopolymers; 44:269-282).

However, other than certain viruses, most DNA condensation process in biological systems, especially in bacteria and eukaryocyte cells, all involve much more complex DNA interactions with large molecules of proteins, and relevant biological information that can be revealed by simple small condensing agents is rather limited. Simple small agents are also unlikely to provide sufficient stabilization and protection of DNA for practical applications. Furthermore, as condensing materials become more complex, elucidation of their complexation process with DNA becomes increasingly difficult. Polymeric condensing agents, for example, have attracted tremendous attention in recent years, due to their superior ability in compacting and stabilizing DNA, and to their chemical flexibility in functional modifications that can improve gene delivery efficiency (Wagner et al., 1990, PNAS; 87:3410-3414; Choi et al., 1998, Bioconjugate Chem; 9:708-718; Mislick et al., 1995, Bioconjugate Chem; 6:512-515; Zauner et al., 1998, Adv Drug Delivery Rev; 30:97-113; Kataoka et al., 1996, Macromolecules; 29:8556-8557; and Kakizawa and Kataoka, 2002, Adv Drug Delivery Rev; 54:203-222). However, interactions between long polymer chains and DNA strands are much more complicated (Nayvelt et al., 2007, Biomacromolecules; 8:477-484), and the innate polydispersity of synthetic polymers further complicates the DNA complexation process. Lack of systematic understanding and precise control about their DNA condensation process represents a severe drawback. In addition, most of the recent DNA condensing materials development is largely driven by application purposes, and not much thought or effort has been put to gaining insights into the DNA condensation process (Wagner et al., 1990, PNAS; 87:3410-3414; Choi et al., 1998, Bioconjugate Chem; 9:708-718; Mislick et al., 1995, Bioconjugate Chem; 6:512-515; Zauner et al., 1998, Adv Drug Delivery Rev; 30:97-113; and Kataoka et al., 1996, Macromolecules; 29:8556-8557).

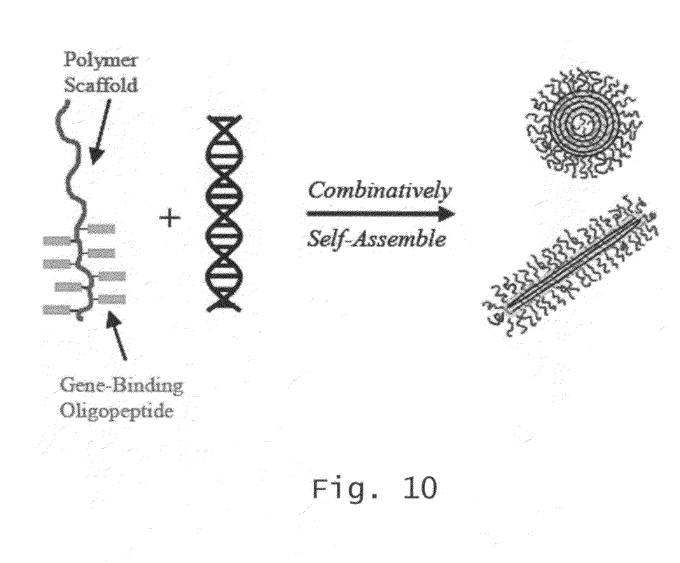

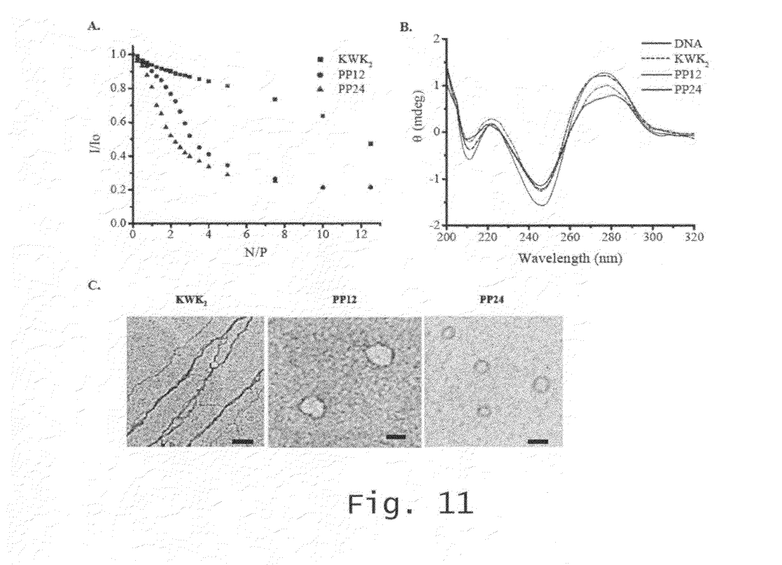

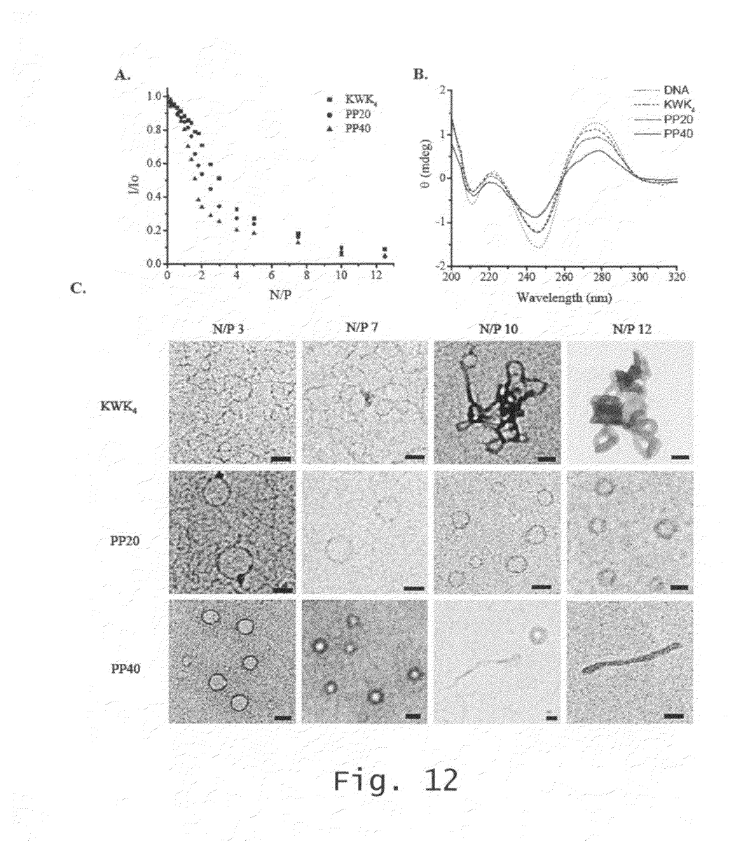

As described in Example 2, a bio-inspired combinative self assembly approach efficiently condenses and packages DNA into nanoparticles (see also, Haley et al., 2008, Mol BioSyst; 4:515-517). Small oligopeptides that emulate the active nucleotide binding site of DNA compaction proteins can provide direct insights into DNA-protein interactions, and when compared to large whole proteins, have the advantages of high efficiency in functionality, less antigenicity, flexibility, and precision in the sequence design (Sparrow et al., 1998, Adv Drug Delivery Rev; 30:115-131). When grafted onto the hydrophobic segment of a block copolymer scaffold, a clustered spatial arrangement of the peptides is created towards DNA binding (FIG. 10).

Synthetic polymers have been used as scaffolds in the past to create multivalent ligands with controlled density, to probe the mechanism of receptor clustering at cell surface and cell signaling pathways (Cairo et al., 2002, J Am Chem Soc; 124:1615-1619). This example elucidates the effect of controlled peptide clustering on the energetics, size, shape, as well as physical properties of DNA condensation in-vitro and provides insights into the general architectural effect of gene binding proteins on DNA condensation and packaging process. Moreover, the versatility afforded by regulating the clustering density and composition of the peptides may provide a novel design platform for gene delivery applications in the future.

### Materials and Methods

λ-DNA in 10 mM Tris buffer was purchased from New England Biolabs (Ipswich, Mass.). All chemicals and solvents were purchased from Sigma-Aldrich (St. Louis, Mo.). Oligopeptides were synthesized by the standard Fmoc solid phase peptide synthesis procedure, using HOBT, HBTU and DIPEA couplings, followed by N-capping with acetylation and C-capping with amidation. Each peptide was analyzed by ESI-MS and ¹H NMR before grafting. Amphiphilic block copolymer PEG₉₃-b-PBD₁₄, where the subscripts denote the average number of repeating units, was prepared by the sequential living anionic polymerization of 1,3-butadiene and ethylene oxide (Forster and Kramer, 1999, Macromolecules; 32:2783-2785).

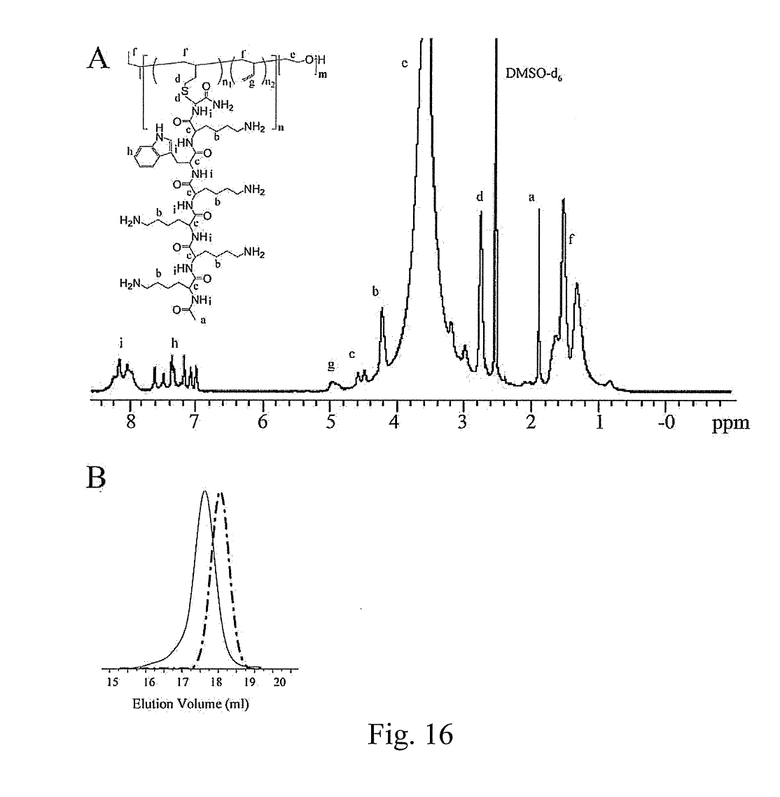

Grafting of model gene-binding KWKₙ peptides onto PEG-b-PBD. For grafting purposes, cysteine that contains a thiol group was attached to KWK oligopeptides as the linker terminus. CKWKₙ were grafted to PEG₉₃-b-PBD₁₄ according to a modular procedure published elsewhere, utilizing the free radical addition of the thiol group onto the double bonds of PBD as described in Example 1 and Example 2 (see also, Haley et al., 2008, Mol BioSyst; 4:515-517; and Geng et al., 2006, Angew Chem, Int Ed; 45:7578-7581). The grafting scheme and the representative NMR and GPC spectra of the polymer-peptide hybrid are shown in FIGS. 15, 16A and 16B. The grafting density, i.e. the percentage of the PBD double bonds grafted with peptides, can be tuned by changing the molar ratio between the reacting thiol groups and double bonds, and the peptides are expected to be randomly linked along the PBD chain (Example 1 and Geng et al., 2006, Angew Chem, Int Ed; 45:7578-7581).

EtBr displacement assay. λ-DNA in Tris buffer (20 μg mL⁻¹) was incubated with EtBr (0.8 μg mL⁻¹) for one hour prior to analysis. The fluorescent intensity of the DNA-EtBr complex was measured using a Jobin Yvon FluoroMax-3 Fluorimeter (excitation: 520 nm, emission: 590 nm). Measured concentrated KWKₙ or their polymer clustered hybrids was then titrated into the DNA-EtBr solution, and the corresponding fluorescence at different lysine/phosphate (N/P) ratios was determined.

Circular dichroism analysis. CD spectra were recorded using Jasco J-715 spectro-polarimeter, at the far-UV region (200-320 nm) and with a scanning speed of 50 nm/min. A total of four scans were accumulated, and temperature was maintained at 25° C. DNA complexes with KWKₙ or their polymer clustered hybrids in Tris buffer were set the concentration of 50 μg/ml and N/P=0.5 for the CD studies. Weak background absorbance from the buffer and condensing agents were directly subtracted from the measurements.

TEM Imaging. λ-DNA complexes with KWKₙ and their polymer clustered hybrids were prepared by mixing an equal volume of DNA (10 mg/mL) with the condensing agents at desired N/P in 1×TE buffer (10 mM Tris-Cl, 1 mM EDTA, pH 7.0). The complex solution was then vortexed for 30 seconds (s) and allowed to equilibrate at room temperature for two hours. The complex sample was then deposited onto the glow discharged formvar coated copper grids and stained with 2% uranyl acetate for one minute. The grids were blotted and then air-dried for TEM imaging on a 200 kV Tecnai 20 transmission electron microscope at a magnification of 10 000×.

DNA melting study. Melting profiles of native λ-DNA and the DNA complexes with KWKₙ or their polymer clustered hybrids (50 μg/mL, N/P=0.5) were obtained by monitoring their absorbance at 260 nm with a Cary 100 UV-Vis spectrophotometer. Samples were heated from 30 to 95° C. with a heating rate of 1° C./min. Weak background absorbance from the buffer and condensing agents were directly subtracted from the measurements.

DNaseI degradation assay. DNaseI (1 unit) in 10× digestion buffer (100 mM Tris-HCl, 25 mM MgCl₂, 5 mM CaCl₂, pH 7.6) was added to 0.02 ml, 10 μg/mL DNA and DNA complex samples (0.2 μg DNA). The samples were incubated at 37° C. for 15 minutes (min), followed by inactivation with 4 μL of 25 mM EDTA at room temperature for 10 min. Finally, 7.5 μL of 100 mg/mL heparin was added and incubated at room temperature for two hours to release DNA for gel electrophoresis analysis (0.8% agarose gel, 1×TAE running buffer, 0.5 μg/mL ethidium bromide, 100 V, 1 hour).

### Results and Discussion

Model gene-binding oligopeptides, KWKₙ (K=lysine; W=tryptophan), with different numbers of lysine residues (n=2 or 4) were used for this study, and they were controllably grafted onto the hydrophobic polybutadiene segment of an amphiphilic poly(ethylene glycol)-block-polybutadiene (PEG₉₃-b-PBD₁₄) block copolymer scaffold at different grafting densities, i.e. either with four peptide grafts or eight peptide grafts, via an established modular peptide grafting route (Example 1 and Geng et al., 2006, Angew Chem, Int Ed; 45:7578-7581). The peptide grafted polymer hybrids are designated as PP series, where PP12 and PP24 represent the polymer-peptide hybrids with four and eight KWK₂ grafted, respectively, and PP20 and PP40 represent the polymer-peptide hybrids with four and eight KWK₄ grafted, respectively. Literature shows that KWKₙ peptides bind to DNA through two kinds of interactions: electrostatic neutralization between the positively charged amino group of lysine (N) and the negatively charged phosphate DNA backbone (P), and the hydrophobic intercalation of aromatic tryptophan within the DNA base pairs (Porschke and Ronnenberg, 1981, Biophys Chem; 13:283-290; and Mascotti and Lohman, 1993, Biochemistry; 32:10568-10579). Such attractions between KWKₙ peptides and DNA are expected to be the driving force for the complexation of the polymer-peptide hybrids with DNA, as control experiments reveal negligible interactions between DNA and the neutral block copolymer scaffold alone (Example 2 and Haley et al., 2008, Mol BioSyst; 4:515-517). Five or more lysine residues are generally required in an oligopeptide sequence to condense DNA (Sparrow et al., 1998, Adv Drug Delivery Rev; 30:115-131; and Wadhwa et al., 1997, Bioconjugate Chem; 8:81-88). With only three lysine residues, free KWK₂ alone exhibited fairly low DNA binding affinity from the ethidium bromide (EB) displacement assay (FIG. 11A). In the EB displacement assay, binding of an agent to DNA would displace the intercalated EB and subsequently quench the fluorescence caused by the EB DNA complex. FIG. 11A shows that free KWK₂ can only weakly quench fluorescence over a wide range of N/P values. Even in large excess, at N/P=12, only 40% of quenching (I/I0 B 0.6) could be achieved by free KWK₂. However, when KWK₂ was clustered into proximity by the polymer PEO₉₃-b-PBD₁₄ scaffold, PP12 and PP24 quenched the fluorescence much more efficiently at the same stoichiometric N/P of free KWK₂, and both were able to achieve nearly complete quenching (I/I0 r 0.3) (FIG. 11A). At higher grafting density, where more oligopeptides were clustered into closer proximity along the polymer scaffold, PP24, with eight peptides grafted, demonstrated more enhanced DNA binding than PP12, with four peptides grafted. It seems that the clustered oligopeptide array gathered by the polymer scaffold can recognize the DNA double helix in a positive cooperative manner and thus can significantly strengthen the DNA binding. The surrounding overall hydrophobic environment generated by the PBD polymers may also contribute to the strengthened DNA binding effect.

Conformational changes in the DNA double helix induced by the binding of KWK₂, PP12 and PP24 were monitored by circular dichroism (CD), FIG. 11B. The CD spectrum of native λ-DNA shows a typical B-form conformation, which is composed of four major peaks in the UV-Vis region: a negative 210 nm peak, a positive 220 nm peak, a negative 245 nm peak and a positive 280 nm peak (Baase and Johnson, 1979, Nucleic Acids Res; 6:797-814). Transition of the DNA conformation from the B-form to the less compact C-form, which is characterized by a decrease in the intensity of the positive 280 nm peak, is commonly found in condensed DNA systems, such as in virus heads and nucleosomes (Baase and Johnson, 1979, Nucleic Acids Res; 6:797-814; and Bottcher et al., 1998, J Am Chem Soc; 120:12-17). With free KWK₂, negligible intensity change at the 280 nm peak was observed and the B-form DNA conformation largely remained intact. PP12 and PP24, on the other hand, provoked much more significant change in the 280 nm peak, indicating a partial B-to-C transition has occurred in such polymer-peptide clustered systems, and the higher the clustering density, the more dramatic the conformational change. It seems that the clustered peptide-DNA binding can cooperatively distort and loosen the DNA double helix into the less compact C form, which facilitates DNA condensation.

Transmission electron microscopy (TEM) analysis reveals distinctively different DNA complexation phenomena between free KWK₂ and its polymer clustered PP12 and PP24 (FIG. 11C). No DNA compaction, but rather exclusively extended DNA bundles, was observed for free KWK₂, even in large excess of N/P of 12. The bundles are 10-20 nm thick and each contains 5-10 λ-DNA strands, considering the individual ds-DNA is −2 nm in width. With just three lysines, free KWK₂ can not compact DNA, but its triple valency is able to bridge different DNA strands together. In sharp contrast, when KWK₂ was clustered by the polymer scaffold, PP12 and PP24 were observed to condense DNA into toroidal structures, and the clustering density exhibited a strong effect on the condensation process. With low clustering density, PP12 gave rise to ill defined nucleation loops with an average large diameter of 80 nm at the same stoichiometric N/P 12. However, no subsequent winding of DNA strands around the nucleation loops, i.e. toroid growth, was fostered, as floppy DNA strands surrounding the loops are clearly visible from the TEM images. As the peptide clustering density doubles in PP24, the toroid loop size was notably reduced and the subsequent toroid growth efficiently promoted. TEM analysis shows well defined DNA toroids that have the average diameter of 50 nm and thickness of 20 nm at N/P=12.

It is well known that the formation of DNA toroid condensates proceeds through two stages, the initial nucleation loop stage, followed by growth (Hud and Vilfan, 2005, Annu Rev Biophys Biomol Struct; 34:295-318). Looping of a semiflexible DNA chain is a spontaneous, diffusion-limited process, and the ease and size largely depend on the flexibility and bending energy of the DNA chain, as well as the ability of the condensing agents to anneal and stabilize the loop (Jun et al., 2003, Europhys Lett; 64:420-426). The cooperative, clustered binding of KWK₂ seems to be able to significantly lower the bending energy of DNA and promote the formation of nucleation loops. With low grafting density, PP12, however, can not sufficiently reduce the DNA helix-helix association energy to foster the further winding of DNA strands around the loop. As the clustering density increases, PP24 can not only overcome the extra strain associated with the smaller nucleation loops, but is also sufficient to promote the further growth of toroids. It appears that for oligopeptides with weak DNA affinity, clustered binding shifts the DNA complexation process from intermolecular bridging to intramolecular toroidal compaction, and the clustering density has a strong impact on the energetics and dimension of the DNA condensation process.

To reveal the effect of clustered binding on DNA condensation for peptides with strong DNA affinity, KWK₄ with five lysine residues and its corresponding polymer clustered hybrids, PP20 (with four peptides grafted) and PP40 (with eight peptides grafted, were studied), respectively. Incorporation of more lysine residues is well known to enhance the binding between the peptide and DNA (Wadhwa et al., 1997, Bioconjugate Chem; 8:81-88). Indeed, EB assay shows that KWK₄ quenched the fluorescence much more efficiently than KWK and reached near complete binding as N/P increased to 10 (FIG. 12A). CD analysis also shows that the binding of KWK₄ to DNA notably reduced the intensity of the 280 nm peak and induced a partial B-to-C conformational change in λ-DNA (FIG. 12B). Like KWK₂, clustering by the polymer scaffold nonetheless enhanced the DNA binding and provoked more pronounced B-to-C conformational change, and the higher the clustering density, the stronger the effect. TEM studies confirmed that free KWK₄ is able to condense DNA into compact structures (FIG. 12C). Intertwined aggregates of toroids were observed as the primary DNA condensate structure. It appears that at initial low N/P values, KWK₄ can simultaneously promote both intermolecular DNA aggregation and the formation of toroid nucleation loops, which progressively grew into intertwined full toroids. When KWK₄ was clustered by the polymer scaffold, PP20 produced discrete toroids without aggregation.

Distinct nucleation loops with an average diameter of 70 nm were observed at N/P=3, and higher N/P values further reduced the toroid loop size and promoted toroid growth. At N/P=12, dispersed, well-defined toroids with average diameter of 45 nm and thickness of 15 nm were found as the exclusive DNA condensate structure. Compared to free KWK₄, which does not seem to differentiate intramolecular DNA compaction from the intermolecular DNA association process, the polymer-peptide clustered PP20 must be able to lower the bending energy of DNA chains much more efficiently, so that it exclusively favors the intramolecular toroidal DNA condensation route. With higher clustering density in PP40, the difference in DNA condensation process becomes even more dramatic from the free KWK₄. PP40 produced much smaller toroid loops with average diameter of 50 nm at N/P=3, and fostered fast toroid growth to 25 nm in thickness at N/P=7. Starting from N/P=10, rod-like DNA condensate structures began to emerge, and upon reaching N/P=12, a significant population (˜50%) of well-defined rods with average length of 200 nm and width of 15 nm were observed. The rods were quite stable in solution and there was no apparent population change with time. This is surprising, considering studies with small condensing agents in literature suggest that DNA rod condensates are generally unstable, and would quickly convert to toroids with time. Controlling the morphology of DNA condensates between toroids and rods has been quite difficult (Vilfan et al., 2006, Biochemistry; 45:8174-8183), and only in the presence of an alcohol solvent that destabilizes the DNA double helix, or with special bacterial chromatin proteins that can induce pronounced kinks in the DNA double helix, has higher population of rods been reported (Arscott et al., 1995, Biopolymers; 36:345-364; and Sarkar et al., 2007, Nucleic Acids Res; 35:951).

Here, the finding of a significant population of stable rods induced by PP40 suggests that at sufficiently high density, the cooperative, clustered binding can sharply bend the DNA double helix into rod-forming kinks, as well as help stabilize the rods once formed. The entanglement nature of the polymer scaffold may also contribute to the stabilization of the rod DNA condensates.

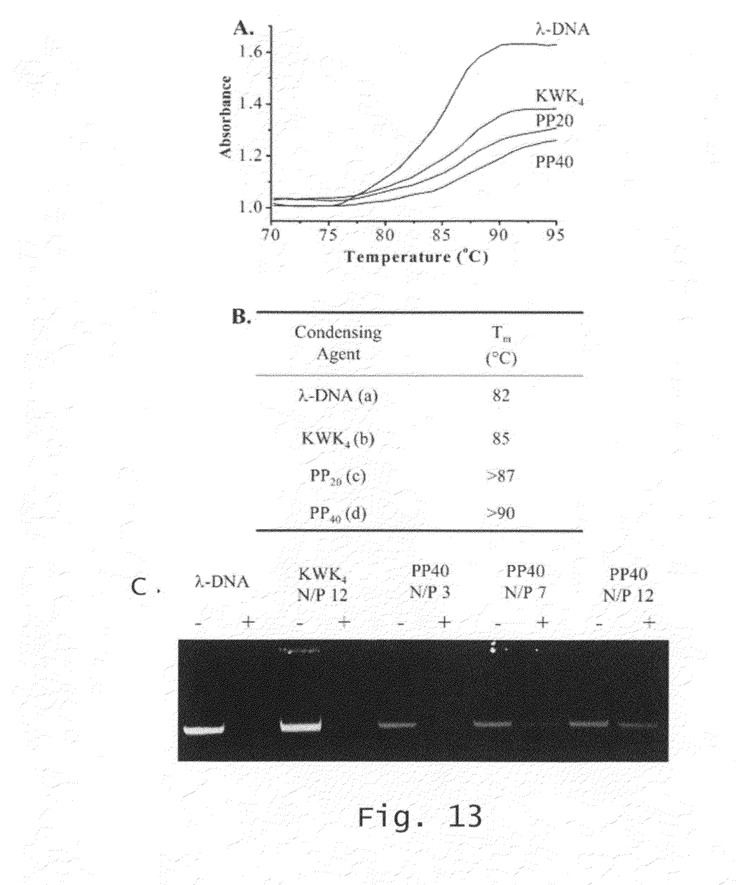

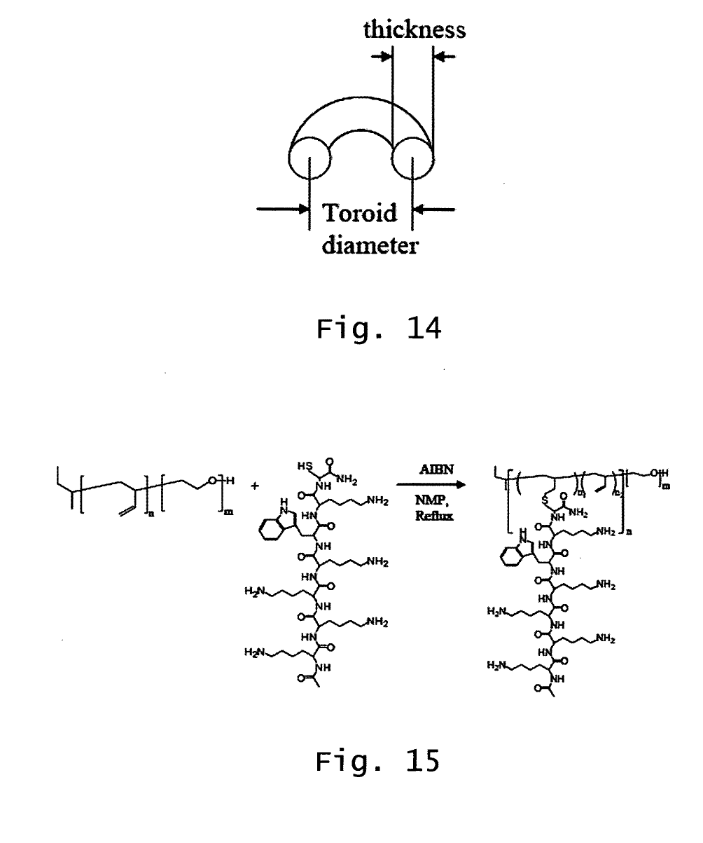

Thus, in the event of oligopeptides with strong DNA affinity, such as KWK₄, this example shows that peptide clustering by a polymer scaffold can alter the DNA condensation pathway from intertwined toroid aggregates to discrete toroid or rod DNA condensates. Controlled density plays a vital role in the clustering effect, which not only determines the size and dimension of DNA condensate structure, but also the shape transition from toroids to rods. To evaluate the effect of the polymer scaffolded peptide clustering on physical properties of the resultant DNA condensates, melting and nuclease degradation studies were carried out to analyze their thermal and biological stability, respectively (FIG. 13). In the melting study, dissociation of double-stranded DNA into single strands was monitored by an increase in the absorbance of 260 nm, due to the disruption of hydrogen bonds between base pairs with raising temperature (i.e. hyperchromic effect). FIGS. 13A and 13B show that compared to naked DNA, condensation with KWK₄ notably shifted the DNA dissociation curve to higher Tm, the temperature at which 50% ds-DNA dissociates, and reduced the degree of change in absorbance, indicating enhanced DNA stabilization against double-strand breakage. When clustered by the polymer scaffold, PP20 and PP40 further stabilized DNA in comparison to free KWK₄, by shifting the DNA dissociation curve to even higher temperatures and more reduced absorbance changes, and the higher the clustering density, the more significant the enhancement. Even at the upper limit of DNA melting studies in aqueous solution, i.e. 95° C., which is just below the boiling point of water, the majority of the ds-DNA remained intact.

DNA is also prone to nuclease degradation in biofluids, which represents a major challenge for gene delivery. To assess their resistance against DNase degradation, naked λ-DNA, DNA condensates with KWK4 and the polymer-peptide clustered PP40, were incubated with DNaseI for 30 min, and the integrity of the DNA before and after the treatment was analyzed by agarose gel electrophoresis (FIG. 13C). For naked λ-DNA, no intact DNA band could be detected after Dnasel treatment, indicating that the DNA has been completely degraded into small fragments that are beyond the detection limit. For DNA-KWK₄ condensates, even at high N/P=12, only a faint intact DNA band was observed after Dnase treatment. Comparison between the DNA band intensity before and after the DNase treatment shows that only a small fraction of DNA was preserved, and KWK₄ itself does not provide sufficient protection for DNA. However, the polymer-peptide clustered PP40 demonstrated much more enhanced DNA protection against nuclease degradation. As more PP40 was used in DNA condensation, the intensity of the intact DNA band after treatment continuously increased with N/P, and at N/P=12, the DNA band before and after DNase treatment was measured to be nearly the same, suggesting that the integrity of the DNA has been largely preserved. It is likely that the superior protection of the block copolymer-peptide clustered hybrids originates from two aspects—highly efficient DNA compaction by the clustered peptides inside the core, and the surrounding dense, stealthy PEG shell that prevents the deposition and degradation of the nuclease. Apparently, the polymer-peptide clustered hybrids here have inherited all the general advantages in DNA stabilization and protection that are associated with polymeric systems, and the PEG shell can be further conjugated with a wide variety of functional groups to foster specific targeting, endosomal release and nuclear transport for future gene delivery applications.

In conclusion, this example demonstrates that peptide clustering can controllably alter the pathway and morphology of DNA condensation in vitro. Moreover, such peptide clustering by block copolymer scaffolds also significantly improves the DNA stability against breakage and DNase bio-degradation. This example has comprehensively elucidated the general architectural effect of the clustered peptide binding on DNA condensation, as well as having provided a versatile new approach to tailor and optimize synthetic gene delivery vector design.

Example 3 has also published as “Effect of clustered peptide binding on DNA condensation” Haley et al., Mol. Biosyst. 2010; 6(1):239-45 (Epub 2009 Sep. 25; DOI: 10.039/008873c) and its accompanying online supporting information all of which are hereby incorporated by reference herein, in their entirety.

### Example 4

### Role of DNA in In-Vitro Condensation

This example reveals the vital role of DNA topology and conformation in directing the combinative self-assembly and condensation pathway and morphology. The phenomenon of DNA condensation into nanostructures has long been studied as a model system to reveal the common principles underlying gene packaging in biology (Gosule and Schellman, 1976, Nature; 259:333). It is also the critical first step towards the development of artificial gene vectors, as studies show that the size and shape of DNA condensates strongly influence their transfection efficiency (Luo and Saltzman, 2000, Nat Biotechnol; 18:33; Molas et al., 2002, Biochim Biophys Acta, Gen Subj; 1572:37; and Stanic et al., 2008, Biomacromolecules; 9:2048). Moreover, condensation can be potentially used in the broader DNA nanotechnology. In recent years, intensive effort has focused on developing synthetic materials for DNA complexation and delivery (Mintzer and Simanek, 2009, Chem Rev; 109:259; Breitenkamp and Emrick, 2008, Biomacromolecules; 9:2495; and Srinivasachari et al., 2008, J Am Chem Soc; 130:4618). Few studies, however, sought to elucidate the role that DNA itself may play in the condensation process (Bronich et al., 2000, J Am Chem Soc; 122:8339; and Arscott et al., 1990, Biopolymers; 30:619). Although limited studies show that DNA topology and conformation can be recognized in complexation processes, the systematic functional role of DNA in determining the condensation pathway and morphology remains unclear.

As shown in the previous examples, a combinative self-assembly approach can efficiently condense and package DNA. An amphiphilic block copolymer was used as a scaffold to create a clustered array of small gene-binding peptide grafts that emulate the active binding site of gene-compaction proteins. Such polymer-peptide hybrids are able to combinatively self-assemble with DNA molecules and condense DNA into nanostructures. This bio-inspired approach can not only reveal insights into the relevant DNA condensation processes found in nature, but it can also provide a versatile design platform for developing artificial gene vectors as well as DNA-based multi-component supramolecular assemblies. See, Example 2; Haley et al., 2008, Mol BioSyst; 4:515; Example 3; and Haley et al., 2010, Mol BioSyst; 6:239.

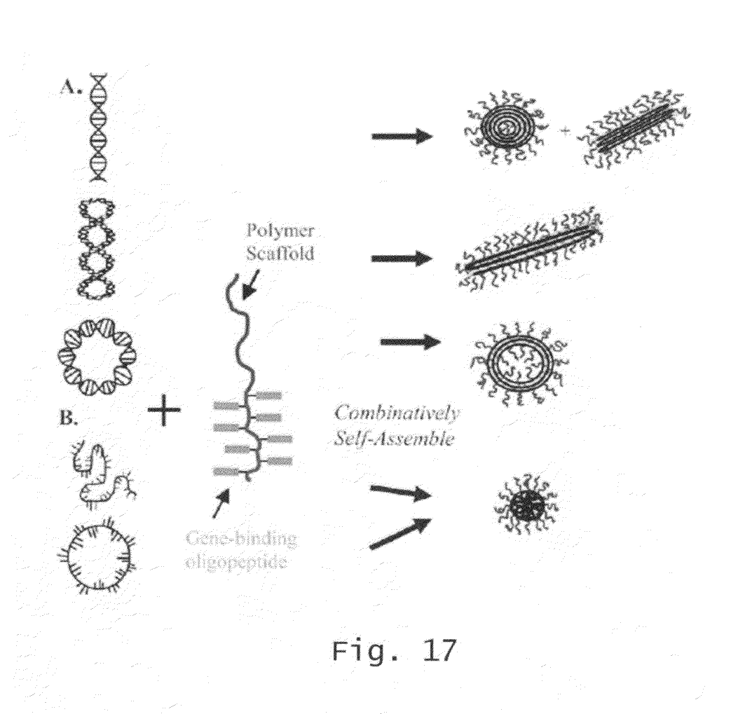

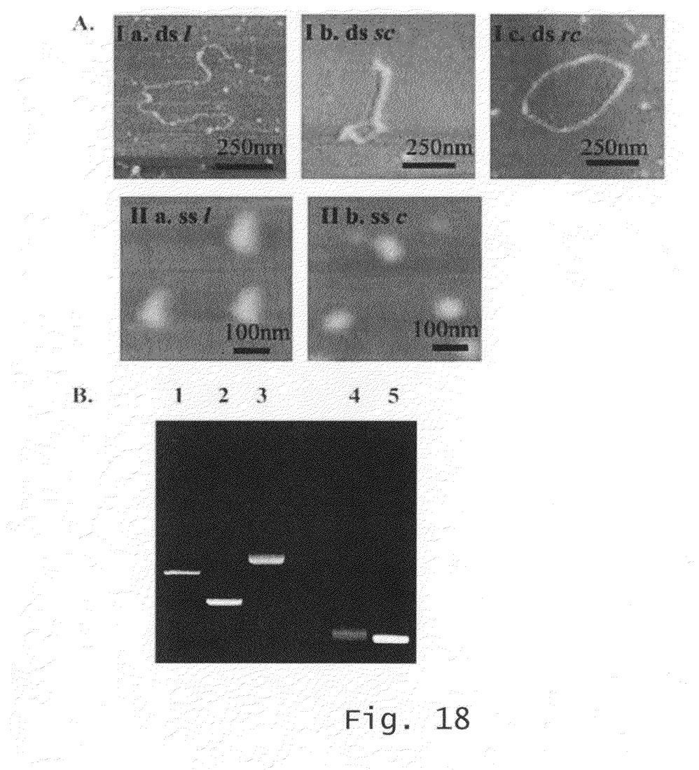

In this example, a new direction reveals the vital role of DNA itself in condensation and combinative self assembly by systematically investigating how DNA topology and conformation affect the pathway and morphology of such processes (see FIG. 17). Model ΦX174 plasmid DNA (4K bp) in five different forms (double-stranded (ds) linear (1), negative-supercoiled (sc) and relaxed-circular (rc), as well as single-stranded (ss) linear and circular (c)) were used for this study. The distinct conformation of each DNA topology was directly visualized by AFM (FIG. 18A). The semi-flexible linear ds-DNA (persistence length Lₚ˜50 nm, 150 bp) exhibited a typical worm-like chain conformation with contour length of 1.5 μm (FIG. 18A (see Ia)). In comparison, a tighter, writhed conformation was observed for the supercoiled ds-DNA, and an open circle conformation for the relaxed circular ds-DNA (FIG. 18A (see Ib and Ic)). Compared to ds-DNA, ss-DNA is much more flexible and contractile (L_{P}˜5 nm, 3 bp) and tends to coil into much tighter structures. Irregular triangular and globular structures of ˜100 nm in size were observed for linear and circular ss-DNA respectively (FIG. 18A (see IIa and III)).

The conformational differences between the five forms of DNA were also reflected in the agarose gel electrophoresis experiments (FIG. 18B), in which the mobility of DNA molecules is primarily determined by their radius of gyration (Aaij and Borst, 1972, Biochim Biophys Acta, Nucleic Acids Protein Synth; 269:192-200). For ds-DNA, the tightest supercoiled ds-DNA migrated the fastest (lane 2), whereas the open relaxed-circular ds-DNA with the biggest cross-section migrated the slowest (lane 3). For ss-DNA, both linear and circular (Lane 4, 5) migrated much faster than ds-DNA due to their much more compact conformations, and only trivial mobility differences were observed between the two forms of ss-DNA due to their similar small sizes.

Each form of ΦX174 DNA was then complexed with a representative polymer-peptide hybrid PP40 to reveal the effect of DNA on the condensation process. PP40 has eight gene-binding oligopeptides KWK₄ (K: lysine; W: tryptophan) grafted onto a polybutadiene-block-poly(ethylene oxide) scaffold, PBD₁₄-b-PEO₉₃, and can efficiently bind with DNA (Examples 2 and 3). Under the same complexation conditions at N/P=3 (N: nitrogen; P: phosphorus), where binding of PP40 with each DNA is complete (Forster and Kramer, 1999, Macromolecules; 32:2783-2785)), different condensation behavior was observed for different forms of DNA (FIG. 19). Depending on the topology, ds-DNA preferably condenses into toroids or rods, whereas the highly flexible ss-DNA generates extremely small spherical nanoparticles.

Statistical analysis on TEM images shows a near 50:50 mixture of toroids and rods for the linear ds-DNA condensates (FIG. 19 (see Ia)). The toroids have well-defined inner holes of 10 nanometer (nm) average diameter and 20 nm thickness, whereas the rods have similar width but 100 nm average length. It is known that semi-flexible ds-DNA strands pack in hexagonal arrays either in loops for toroids or in bundles for rods (Bloomfield, 1991, Biopolymers; 31:1471). The equal population of toroids and rods for linear ds-DNA suggests that, when condensing with an agent that can efficiently stabilize the sharp bends of ds-DNA in the rod formation (e.g. PP40) (see Examples 2 and 3), packing of ds-DNA in rods and toroids is virtually isoenergetic and these two morphologies can coexist at equilibrium (Bloomfield, 1991, Biopolymers; 31:1471). In comparison, negative-supercoiled ds-DNA predominantly condenses into rods that appear to be slightly thinner and longer than that of linear ds-DNA (FIG. 19 (see Ib)). This is likely because negative supercoiling unwinds the DNA double helix and facilitates sharp bending to occur, thus enhancing the ease of rod formation. Without negative supercoiling, the relaxed-circular ds-DNA predominantly condenses into thin toroids with large holes instead, which have an average thickness of 8 nm and inner hole diameter of 35 nm (FIG. 19 (see Ic)). The notable differences between the relaxed circular and linear ds-DNA toroids indicate that the topological strain associated with the enclosed circular ds-DNA has a significant impact on toroid formation. Toroids are known to form through two steps: spontaneous nucleation loop formation followed by toroidal growth, i.e. subsequent winding of DNA strands around the nucleation loop (Conwell et al., 2003, PNAS; 100:9296). The nucleation loop size is sensitive to the flexibility of the DNA (Jun et al., 203, Europhys Lett; 64:420), and we expect extra strain from circular ds-DNA to enlarge the loop size. Extra strain by circular ds-DNA can also alter the subsequent toroidal growth pathway. While linear ds-DNA can grow freely both outward and inward from the nucleation loop, with the inward growth diminishing the hole size (Conwell et al., 2003, PNAS; 100:9296), the circular ds-DNA seems to prefer the less strained outward growth pathway, leading to thin toroids with large holes.

For highly flexible and contractile ss-DNA, packing into hexagonally ordered toroids or rods is clearly not energetically favored. Significant tightening from the initial 100 nm coiled structures to extremely small nanoparticle condensates of −15 nm in size was observed for both linear and circular ss-DNA instead (FIG. 19 (see IIa and IIb). Although at equal N/P ratio, the number of polymer chains relative to the ss-DNA strands would be half that of the double-stranded DNA, the tendency of ss-DNA to condense into small nanoparticles seems to be determined by its highly flexible and contractile nature, not by this polymer chain-DNA strand ratio difference. Even when more PP40 was used and the N/P ratio doubled, small nanoparticle condensates from ss-DNA were still observed.

In summary, this example elucidated the systematic functional role of DNA in directing the combinative self-assembly and condensation pathway and morphology. This discovery highlights the significance of the gene itself in the condensation process, will lead to new strategies for designing artificial gene vectors, and shed light on the diverse kinds of viral gene packaging found in nature. For example, the tendency of ss-DNA to condense into such small particles may well be correlated to the structure of ss-DNA viruses, such as the adeno-associated virus, which is one of the smallest yet most infectious mammalian viruses. Moreover, exploring the role of DNA in combinative self-assembly should inspire new directions for fabricating multi-component supramolecular assemblies.

### Materials and Methods

The double-stranded negative-supercoiled, relaxed-circular and single-stranded circular ΦX174DNA, supplied in 1×TE buffer pH 8.0, were purchased from New England Biolabs. The 1×TE buffer was exchanged for 18.2 MΩ water using Qiagen QIAquick gel extraction kit. The quantity of the DNA was determined by spectrophotometric analysis at 260 nm. DNA was diluted to a final concentration of 20 ug/mL in 18.2 uΩ water for further studies.

Obtaining the linear double-stranded DNA by linearization of the negative-supercoiled ΩX174 plasmid DNA. Double-stranded supercoiled ΩX174 plasmid DNA was linearized with the restriction enzyme SspI and purified using Qiagen QIAquick gel extraction kit according to supplier's protocol. Briefly, five units of SspI/ug DNA in 1× NEBuffer were incubated at 37° C. for three hours, followed by inactivation at 65° C. for 30 minutes. Three volumes of Buffer QC and one volume of isopropanol was added to the reaction mixture, placed in the spin column, centrifuged and washed with Buffer PE. Linearized DNA was eluted from the column with 18.2 uΩ water. Full linearization of the DNA was determined by agarose gel electrophoresis (1.2% agarose, 100V, 60 minutes).

Obtaining the linear single-stranded DNA by denaturation of the linear double-stranded DNA. Denaturation of the linear double-stranded DNA was performed by heating the DNA solution to 95° C. for four minutes, followed by immediate incubation on ice. The degree of denaturation of the single-stranded linear ΦX174 RFI was assayed by agarose gel electrophoresis (1.2% agarose, 100V, 60 minutes). The quantity of double-stranded linear ΦX174 RFI and single-stranded linear ΦX174 RFI was determined by spectrophotometric analysis at 260 nm. The DNA was diluted to a final concentration of 20 ug/mL in 18.2MΩ water for further studies.

Grafting of gene-binding oligopeptide onto PEG-b-PBD. The amphiphilic diblock copolymer scaffold (PBD₁₄-b-PEO₉₃, subscripts denote the average number of repeating units) with a narrow molecular-weight distribution (polydispersity index, PDI=1.05) was prepared by the well-established sequential anionic polymerization of buta-1,3-diene and ethylene oxide (Forster and Kramer, 1999, Macromolecules; 32:2783-2785). Thiol-containing Cysteine (Cys) was attached to the gene-binding oligopeptide sequence KWK₄ as the linker terminus for grafting purpose. Peptide CKWK₄ was synthesized using the standard Fmoc Solid Phase Peptide Synthesis procedure with HOBT, HBTU, and DIPEA couplings, followed by N-capping with acetylation and C-capping with amidation. The synthesized peptides were analyzed by ESI-MS and ¹H NMR before grafting. CKWK₄ was then grafted to PBD₁₄-PEO₉₃ to produce PP40 according to the procedure published elsewhere, utilizing the free radical addition of the thiol group of the cysteine to the double bonds of PBD (Example 1; Geng et al., 2006, Angew Chem; 45:7478-7581; Example 3; and Haley and Geng, 2010, Mol BioSyst; 6(1):239-45).

Agarose Gel Electrophoresis. Fifteen microliters of each DNA sample was loaded onto a 1.2% agarose gel, and the electrophoresis was ran at 100V for 60 minutes in 40 mM Tris-acetate, 1 mM EDTA, pH 8 running buffer. Gels were stained with ethidium bromide (0.5 ug/ml, 60 minutes) and visualized with a UV transilluminator.

AFM Analysis. 10 ug/mL of each DNA aqueous solution was mixed 1:1 with 20 mM Tris, 2 mM EDTA, 12.5 mM MgCl₂. Two microliters of the mixed DNA solution was then deposited on to freshly cleaved mica and dried under a gentle flow of nitrogen gas. AFM imaging was then performed using tapping mode at ambient temperature on a Dimension 3100 AFM (Veeco), equipped with Nanoscope III software (Digital Instruments, Santa Barbara, Calif.). Silicon probes (Veeco RTESP, spring constant 40N/m, resonant frequency 300 Hz) were used to obtain all images.

TEM Analysis. Equal volume of measured PP40 (78 μM was added to each DNA aqueous solution (10 ug/ml) to obtain final N/P ratio of 3 (N: nitrogen from positively charged lysine; P: phosphorous from the negatively charged DNA backbone). The DNA-PP40 complexes were allowed to equilibrate at room temperature for one hour and then deposited onto glow discharged formvar coated copper grids. The complexes were then stained with 2% uranyl acetate for one minute, and the grids were blotted and air-dried. EM imaging was performed on a 200 kV Tecna 20 transmission electron microscope at a magnification of 10,000×.

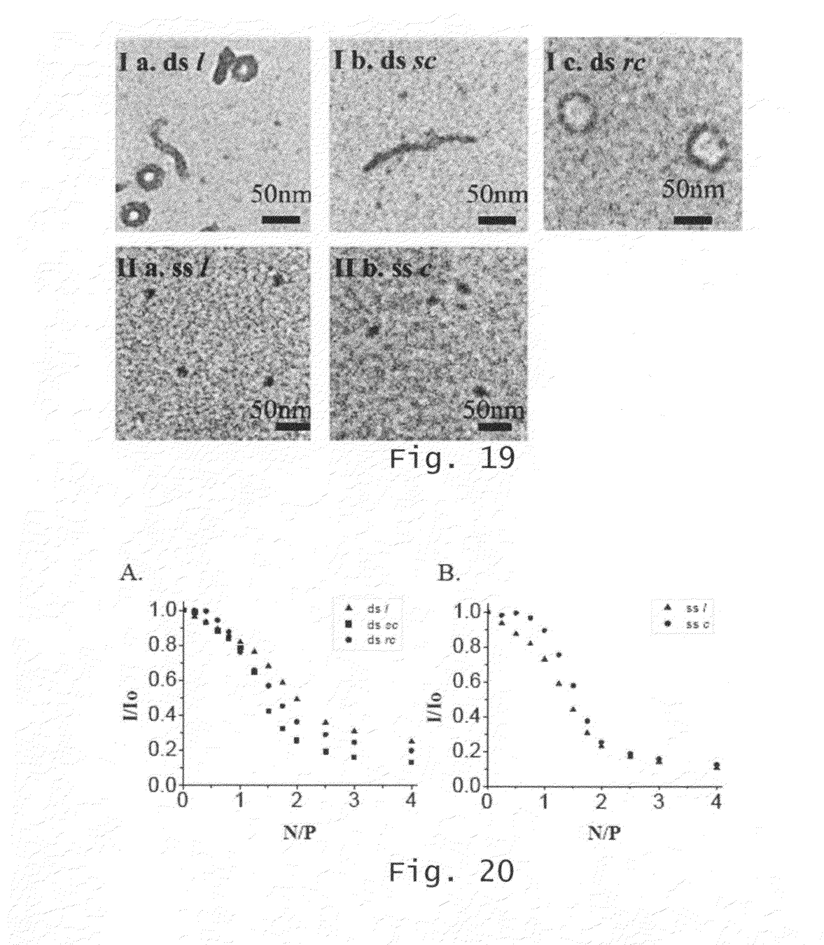

Ethidium bromide (EB) displacement assay. The degree of DNA condensation was determined as a function of the N/P ratio by a ethidium bromide displacement assay. In the EB displacement assay, binding of an agent to DNA would displace the intercalated EB and subsequently quench the fluorescence caused by the EB-DNA complex. Before measurement, DNA (20 ug/mL) was incubated with EtBr (0.8 ug/mL) for one hour. Concentrated PP40 (78 uM) was then titrated into the DNA-EtBr solution. The fluorescence intensity of samples at different N/P ratios were excited at 520 nm, and the fluorescence was measured at 590 nm at temperature of 25° C., using a Jobin Yvon FluoroMax-3. Sample fluorescence was determined after subtracting the baseline fluorescence of EtBr in the absence of the DNA. See FIG. 20.

Example 4 has also published as “Role of DNA in condensation and combinative self-assembly,” Haley et al., 2010, Chem Commun (Camb); 46(6):955-7 (Epub 2009 Dec. 23; DOI: 10.1039/b9214040 and its accompanying online supporting information, each of which are hereby incorporated by reference herein, in their entirety.

### Example 5

### Structural Effect of the Peptide-Polymer Hybrid on Condensation Process

In order to fully understand the structural effect of the polymer-peptide hybrid in determining the combinative self-assembly DNA condensation process, this example will systematically investigate both of the specific effect of the small peptide and the architectural effect of the block copolymer scaffold on DNA condensation mechanism, pathway, morphology (such as, for example, size and shape) and physical properties (such as, for example, thermo- and bio-stability).

Aim A of this example will characterize the amino acid structural and sequence specificity of the gene-binding oligopeptide on the combinative self-assembled DNA condensation. The oligopeptides of the hybrid molecule that emulate the active binding site of the DNA compaction proteins play a pivotal role in the DNA complexation and condensation of the combinative self-assembly process. Peptides can interact with DNA molecules via a variety of non-covalent forces. While electrostatic attraction, between the positively charged amino acids and the negatively charged phosphate groups of DNA, is the most important factor in the DNA condensation process, other non-covalent interactions, such as hydrogen-binding, π-π stacking and hydrophobic interactions, can also mediate the DNA complexation and condensation process. A thorough investigation on the effect of amino acid structural and sequence specificity on the combinative self-assembly DNA condensation process will provide key information on the peptide-DNA interactions that determine the mechanism, pathway and morphology of DNA condensation. Such information can not only shed insights into the biological protein-DNA compaction processes, but also provide key information for the rational design of peptide sequence towards predicting and controlling DNA condensation.

Experimental Design. Basic amino acids—Arg and Lys: the electrostatic factor. The electrostatic attraction between negatively charged phosphate groups of DNA and the positively charged basic amino acid side chains is the fundamental driving force governing DNA binding and condensation. Arg (pKₐ=12) and Lys (K, pKₐ=10) are two basic amino acids that bear positively charge under physiological condition (pH=7.4), and are rich in DNA compaction proteins, such as histone. Compared to Lys with a primary amino group, Arg has a conjugated guanidinium group at the end, where the positive charge is de-localized. Studies in literature show that Arg has higher DNA binding affinity and can induce DNA condensation more efficiently than Lys. As the fundamental electrostatic factor in the combinative self-assembly DNA condensation process, this example will investigate and compare the structural specificity of Lys and Arg in the DNA-binding oligopeptide sequence of the peptide-polymer hybrid.

OligoLysines and oligoArginines, nK and nR (n=2, 3, 5), with Cysteine as the linking terminus, will be synthesized by SPPS, and then grafted onto the PBD-b-PEO scaffolds of PBD₁₄-b-PEO₉₃ and PBD₂₅-b-PEO₇₅, at different densities, with four and eight grafts respectively. Their DNA binding and condensation process will be studies and compared. The binding affinity of each peptide-polymer hybrid with a model plasmid DNA over a wide N/P range will be studied by EtBr Displacement Assay. Any potential DNA conformational change induced by binding will be monitored by CD. The equilibrated structure of their combinatively self-assembled DNA complexes under different N/P will be characterized by TEM. The stability of each DNA complex against double strand breakage and nuclease degradation will be evaluated by melting and Gel Electrophoresis studies, respectively.

Histidine: the hydrogen-binding factor. Although the electrostatic force is fundamental for DNA condensation, and oligoLysines or oligoArginines will be used as the foundation for peptide sequence design, incorporating other amino acids that enhance DNA binding into the peptide sequence can significantly affect the DNA condensation process.

The imidazole side chain of Histidine (H) has a pKa ˜6, and is mostly neutral at the physiological pH 7.4. However, studies in literature show that the imidazole side chain of Histidine can bind to the minor groove of DNA via multiple hydrogen-bonding with the base pairs. By using the oligoLysines or oligoArginines based polymer-hybrids as controls, this example will evaluate the influence of incorporating Histidine into the peptide sequence of the peptide-polymer hybrid on the combinative self-assembly DNA condensation process. This example will also study the effect of the position of Histidine within the peptide sequence on DNA binding and condensation.

Histidine (H) will be included to both the end and the middle of the oligoLysine or oligoArginine sequences, e.g. KₙH and KHKₙ₋₁, and then grafted onto the PBD-b-PEO scaffolds of PBD₁₄-b-PEO₉₃ and PBD₂₅-b-PEO₇₅, at different densities, with four and eight grafts respectively. This example will thoroughly study and compare their DNA binding and condensation process with the control oligoLysine and oligoArginine based systems. The binding affinity of each peptide-polymer hybrid with a model plasmid DNA over a wide N/P range will be studied by EtBr Displacement Assay. Any potential DNA conformational change induced by binding will be monitored by CD. The equilibrated structure of their combinatively self-assembled DNA complexes under different N/P will be characterized by TEM. The stability of each DNA complex against double strand breakage and nuclease degradation will be evaluated by melting and Gel Electrophoresis studies, respectively.

Tryptophan: the π-π stacking factor. Studies in literature show that the aromatic indole side chain of Tryptophan (W) can intercalate into the DNA, due to π-π stacking with base pairs. By using the oligoLysines or oligoArginines based polymer-hybrids as controls, this example will evaluate the influence of incorporating Tryptophan into the peptide sequence of the peptide-polymer hybrid on the combinative self-assembly DNA condensation process. This example will also study the effect of the position of Tryptophan within the peptide sequence on DNA binding and condensation.

Tryptophan (W) will be included to both the end and the middle of the oligoLysine or oligoArginine sequences, e.g. KₙW and KWKₙ₋₁, and then grafted onto the PBD-b-PEO scaffolds of PBD₁₄-b-PEO₉₃ and PBD₂₅-b-PEO₇₅, at different densities, with four and eight grafts respectively. DNA binding and condensation process will be studies and compared with the control oligoLysine and oligoArginine based systems. The binding affinity of each peptide-polymer hybrid with a model plasmid DNA over a wide N/P range will be studied by EtBr Displacement Assay. Any potential DNA conformational change induced by binding will be monitored by CD. The equilibrated structure of their combinatively self-assembled DNA complexes under different N/P will be characterized by TEM. The stability of each DNA complex against double strand breakage and nuclease degradation will be evaluated by melting and Gel Electrophoresis studies, respectively.

Proline: the effect of the cyclic amino acid. The distinctive cyclic structure of Proline's (P) side chain locks its Φ backbone dihedral angle at approximately −75°, giving Proline an exceptional conformational rigidity compared to other amino acids. Incorporating Proline can cause turns in the peptide sequence, and thus profoundly affect DNA complexations. By using the oligoLysines or oligoArginines based polymer-hybrids as controls, this example will evaluate the influence of incorporating Proline into the peptide sequence of the peptide-polymer hybrid on the combinative self-assembly DNA condensation process. This example will also determine whether the position of Proline within the peptide sequence has an effect on DNA binding and condensation.

Proline (P) will be inserted to both the end and the middle of the oligoLysine or oligoArginine sequences, e.g. KₙP and KPKₙ₋₁, and then grafted onto the PBD-b-PEO scaffolds of PBD₁₄-b-PEO₉₃ and PBD₂₅-b-PEO₇₅, at different densities, with four and eight grafts respectively. This example will thoroughly study and compare their DNA binding and condensation process with the control oligoLysine and oligoArginine based systems. The binding affinity of each peptide-polymer hybrid with a model plasmid DNA over a wide N/P range will be studied by EtBr Displacement Assay. Any potential DNA conformational change induced by binding will be monitored by CD. The equilibrated structure of their combinatively self-assembled DNA complexes under different N/P will be characterized by TEM. The stability of each DNA complex against double strand breakage and nuclease degradation will be evaluated by melting and Gel Electrophoresis studies, respectively.

Expected results. Compared to oligoLysines, the oligoArginine based polymer-peptide hybrids are expected to display enhanced DNA binding affinity and induce more pronounced DNA conformational changes. They are expected to condense DNA much more effectively in the combinative self-assembly process, which should be reflected in the size, shape, and population of the condensate structures under equilibrium, i.e. smaller nucleation loop size and more efficient growth in the toroid formation and enhanced ease in rod formation are expected. The oligoArginine based polymer-peptide hybrids are also expected to produce more stabilized DNA condensates against double strand breakage and nuclease degradation. With the addition of a Trp or a His into the peptide sequence, enhanced DNA binding affinity, more pronounced DNA conformational change, more efficient DNA condensation and better stabilization compared to pure oligoLysine and oligoArginine based polymer-peptide hybrids are expected. The position of the Trp or His within the peptide sequence may also influence their peptide-polymer hybrid DNA condensation. Addition of a Pro expected to significant facilitate sharp bending of DNA, due to the structural turn in the peptide sequence that can be induced by the unique cyclic side chain of Pro. Predominant rod formation is expected.

Aim B of this example will evaluate the architectural effect of the block copolymer scaffold on the combinative self-assembly DNA condensation, addressing both the grafting density and the spacing between peptide grafts. While the peptides are responsible complexing with DNA, the block copolymer scaffold brings the peptides into vicinity and creates a clustered architectural arrangement of the DNA-binding peptides, which exerts a profound impact on combinative self-assembly DNA condensation, as demonstrated in the previous examples. There are two critical parameters associating with the architectural peptide arrangement caused by the polymer scaffold, the number of peptides clustered together (the grafting density), and the spacing between grafted peptides. The previous examples have systematically studied how different grafting density with the same block copolymer scaffold affects the DNA condensation. Aim B will elucidate the effect of peptide spacing on the DNA condensation mechanism, pathway, morphology and physical properties. Such knowledge will be important for optimizing the PBDₘ-b-PEOₙ design towards desired combinative self-assembly DNA condensation.

Experimental Design. A series of PBDₘ-b-PEOₙ with varying length of the PBD block (n=8, 14, 30, 60, 90, 120), synthesized by sequential anionic polymerization, will be used as scaffold for peptide grafting. Although the developed radical grafting route does not predict the exact position for each peptide, and the grafted peptides will be randomly distributed along the PBD segment, the average spacing between peptides can still be tuned by the PBD polymer length, i.e. when the number of the peptide grafts is the same, the longer the PBD polymer, the longer the average peptide spacing will be. At least two oliopeptide sequences designed based on Aim A will be grafted onto each PBDₘ-b-PEOₙ. The number of peptide grafts on each polymer scaffold will be controlled to be the same, either with four peptide grafts or eight peptide grafts. The binding affinity of each peptide-polymer hybrid with a model plasmid DNA over a wide N/P range will then be studied by EtBr Displacement Assay. Any potential DNA conformational change induced by binding will be monitored by CD. The equilibrated structure of their combinatively self-assembled DNA complexes under different N/P will be characterized by TEM. The stability of each DNA complex against double strand breakage and nuclease degradation will be evaluated by melting and Gel Electrophoresis studies, respectively.

Expected results. It is expected that for a certain range of n, the shorter PBD length will cause closer spacing between grafted peptides, and will be more efficient in DNA condensation, where smaller nucleation loop size and more efficient growth in the toroid formation, enhanced ease in rod formation, and improved stability against double strand breakage and nuclease degradation will be expected.

### Example 6

### Role of DNA in the Combinative Self-Assembly Condensation Process

Compared to the tremendous effort on the development of novel DNA condensing materials, not much is known about the role of the DNA molecule itself in the condensation process. As shown in the previous Example 4, the DNA molecule plays a significant role in the combinative self-assembly condensation process. This example will study the role of DNA in condensation by studying the general effect of DNA topology and conformation, the effect of inserted specific nucleotide sequences, and the oligonucleotide condensation, respectively. This example will address critical functional role of DNA in determining the condensation mechanism, pathway and morphology and provide critical information for optimizing gene design towards vector development.

Aim A of this example will elucidate the general effect of DNA topology and conformation on condensation. In the previous examples, by using ΦX174 plasmid DNA in five different forms (linear double-stranded (ds), negative-supercoiled ds, relaxed-circular ds and linear single-stranded (ss), circular ss) and a polymer-peptide hybrid PP40 (PBD₁₄-b-PEO₉₃ scaffold, with eight KWK₄ grafts), it was shown that DNA topology and conformation have a strong influence on DNA condensation pathway and morphology. This example will extend the studies to a variety of plasmid DNAs in various forms and a series of polymer-peptide hybrids with different peptide sequence and grafting density and obtain a systematic understanding on the general effect of DNA topology and conformation on condensation.

Experimental Design. This example will use a variety of plasmid DNA in different forms, including PUC18 plasmid DNA (2686 bp), Bluescript II SK plasmid DNA (2961) and M13 phage plasmid DNA (6500 bp). The natural negatively-supercoiled circular dsDNA of PUC 18 and Bluescript are directly available from biotech companies, and their corresponding relaxed-circular and positive-supercoiled dsDNA forms can also be ordered upon request. The circular dsDNA can then be cut and linearized by restriction enzymes at the selected site. Both linear and circular single-stranded DNA can be obtained by the denaturation of double-stranded DNA. For M13 phage DNA, both the double-stranded and single-stranded circular forms are directly commercially available, and can be further cut by restriction enzymes into linear forms. AFM will be used to directly visualize the conformational difference between each DNA form, and Agarose Gel Electrophoresis will be used to characterize their mobility difference relating to conformation.

A series of polymer-peptide hybrids with different peptide sequence, PBD polymer length and grafting density will then be used to combinatively self-assemble with each different form of DNA. The DNA binding over a wide N/P range will be studied by EtBr Displacement Assay. The equilibrated DNA condensate structure under different N/P will be characterized by TEM and analyzed.

Expected results. Like ΦX174 plasmid DNA, it is expected that different DNA topology and conformation will lead to distinctively different DNA condensation pathway and morphologies. For the semi-flexible double-stranded linear DNA, a near 50:50 mixture of toroids and rods is expected. In comparison, negative-supercoiled dsDNA is expected to preferably condenses into rods, as negative supercoiling unwinds the DNA double helix and facilitates sharp bending of the DNA double helix into rod formation. The relaxed-circular dsDNA is expected to preferably condenses into thin toroids with large inner holes, due to the topological strain associated with the enclosed circular DNA. For the highly flexible single-stranded DNA, both linear and circular ssDNA are expected to further tighten into extremely small spherical nanoparticles.

Aim B of this example will evaluate the special effect of specific nucleotide sequence inserted to DNA molecule on condensation. From studies with simple cations in literature, it is know that some specific nucleotide sequences, when inserted into DNA, are able to alter the curvature or local secondary structure of DNA, and consequently change its condensability. For example, multiple A-tracts, which is a sequence of four to eight consecutive adenine residues, when inserted into plasmid DNA, can generate a static loop to direct toroid nucleation. Inserting alternating d(CG)n, on the other hand, is capable of inducing a local B-to-Z transition in DNA. By thoroughly evaluating the special effect of inserting A-tracts and d(CG)n sequences on the combinative self-assembly condensation process, the information obtained can provide a useful means towards control and fine-tuning of the DNA condensation pathway and morphology.

Experimental Design. A 173-bp A-track sequence 5′-ATCCATCGACC-(AAAAAACGGGCAAAAAACGGC), AAAAAAGCAGTGGA-AGC-3′ (SEQ ID NO: 1) will be synthesized using standard phosphoramidite synthesis on an automated DNA synthesizer. After multiplication with PCR, one, two, three or four tandem copies of the double-stranded A-tracks sequence will be inserted into the Bluescript II SK plasmid DNA, following the reported ligation procedure. A series of polymer-peptide hybrids with different peptide sequence and grafting density will then be used to combinatively self-assemble with the A-tracks inserted Bluescript plasmid DNA in comparison with the native Bluescript plasmid DNAs itself. The dimension of DNA condensate structure under different N/P will be characterized by TEM and analyzed.

One or two copies of a 20-bp alternating d(CG)n sequence (SEQ ID NO:2) will be inserted into the negative supercoiled PUC18 plasmid DNA, following the reported procedure. The potential local B-to-Z transition caused by the inserted d(CG)n sequence will be monitored by CD. A series of polymer-peptide hybrids with different peptide sequence, PBD length and grafting density will then be used to combinatively self-assemble with the d(CG)n sequence inserted PCU18 plasmid DNAs in comparison with the native PUC 18 plasmid DNA itself. The dimension of DNA condensate structure under different N/P will be characterized by TEM and analyzed.

Expected results. With two or more insertion of the 173-bp A-tracks into Bluescript II SK plasmid DNA, a small static loop can be formed and serve as the toroid nucleation loop for further growth. Thus, toroids are expected to be the predominant DNA condensate morphology, and they are expected to have much smaller inner holes compared to the native Bluescript plasmid DNA. With the d(CG)n sequence inserts, they are expected to induce local B-to-Z transition and thus enhance the DNA condensability. A more tightly packed rods are expected for d(CG)n inserted PUC18 plasmid DNA compared to the native negative supercoiled PUC18 plasmid DNA.

Aim C of this example will investigate the oligonucleotide condensation via combinative self-assembly. Oligonucleotides are quickly emerging as important therapeutic genetic materials in the last few years, such as, for example, antisense oligonucleotides, and the advancement in oligonucleotide condensation would be highly desirable for gene medicine. Oligonucleotides with twenty or less base pairs are much shorter compared to plasmid DNA, typically having thousands of base pairs, and the condensation of dispersed short oligonucleotides into compact nanostructures is likely to be drastically different from plasmid DNA. By systematically exploring the condensation of both double-stranded and single-stranded oligonucleotides via the combinative self-assembly, the knowledge gained can further expand the scope and versatility of this approach.

Experimental Design. Single-stranded 20 bp oligonucleotide sequences of alternating d(CG)₁₀ (SEQ ID NO:2), d(AT)₁₀ (SEQ ID NO:3) and dC₂₀ (SEQ ID NO:4), dG₂₀ (SEQ ID NO:5), dA₂₀ (SEQ ID NO:6), dT₂₀ (SEQ ID NO:7), as well as the double-stranded sequences of d(CG)₁₀ (SEQ ID NO:2), d(AT)₁₀ (SEQ ID NO:3), and dC₂₀ (SEQ ID NO:4)-dG₂₀ (SEQ ID NO:5), dA₂₀ (SEQ ID NO:6)-dT₂₀ (SEQ ID NO:7), will be first used to get a fundamental understanding on the oligonucleotide condensation. The oligopeptides will be synthesized by DNA synthesizer. The binding and condensation of each oligopeptide with a series of peptide-polymer hybrids with various peptide sequence, PBD polymer length, and grafting density will be studied by CD and TEM respectively.

Single-stranded antisense oligonucleotides that have potential medical use will then be studies, such as, for example, 5′-ATATTCCGTCATCGC-3′ (SEQ ID NO:8), which is reported to be complementary to part of the first four codons and upstream sequence close to the ribosome-binding site of c-Has-ras mRNA. The binding and condensation of desired antisense oligopeptides with a series of peptide-polymer hybrids with various peptide sequence and grafting density will be studied by CD and TEM respectively.

With the single-stranded oligonucleotides, considering their high flexibility, it is expected that spherical condensates as the predominant condensate structure, and the condensate size can be fine-tuned by the peptide sequence, PBD polymer length and grafting density. With the double-stranded oligonucleotides a diverse and novel condensation phenomenon could be generated.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

All headings are for the convenience of the reader and should not be used to limit the meaning of the text that follows the heading, unless so specified.

Sequence Listing Free Text

| | |
|---|---|
| SEQ ID NO: 1 | 173-bp A-track sequence |
| SEQ ID NO: 2-7 | Twenty base pair synthetic |
| | oligonucleotides |
| SEQ ID NO: 8 | Synthetic oligonucleotide |

What is claimed is:

1. A composition comprising a polymer-peptide hybrid and one or more isolated polynucleotides;
wherein the polymer of the polymer-peptide hybrid comprises a neutral amphiphilic block copolymer;
wherein the peptide of the polymer-peptide hybrid is covalently attached to the hydrophobic block of the amphiphilic block copolymer;
and wherein the one or more isolated polynucleotides are complexed with the polymer-peptide hybrid via electrostatic interactions between the peptide of the polymer-peptide hybrid and the one or more polynucleotides.

2. The composition of claim 1 wherein the amphiphilic block copolymer comprises a diblock copolymer.

3. The composition of claim 1 wherein the hydrophobic block of the amphiphilic block copolymer comprises one or more double bonds available for free radical addition reaction of a thiol group of the peptide.

4. The composition of claim 1 wherein the hydrophilic block of the amphiphilic block copolymer comprises polyethylene glycol (PEG) or poly(ethylene oxide) (PEO).

5. The composition of claim 1 wherein the hydrophobic block of the amphiphilic block copolymer comprises a hydrophobic block selected from the group consisting of polybutadiene (PBD), polyethyl ethylene (PEE), poly(2-isopropyl-2-oxazoline), and combinations thereof.

6. The composition of claim 1 wherein the amphiphilic block copolymer comprises poly(ethylene glycol)-block-polybutadiene (PEG-b-PBD).

7. The composition of claim 6 wherein PBD-b-PEG comprises PBDₘ-b-PEGₙ, wherein n and m are independently about 8 to about 120.

8. The composition of claim 6 wherein PBD-b-PEG comprises PBD₁₄-b-PEG₉₃ or PBD₂₅-b-PEG₇₅.

9. The composition of claim 1 wherein the peptide comprises Kₙ, Rₙ, KₙW, RₙW, KWKₙ₋₁, RWRₙ₋₁, KₙH, RₙH, KHKₙ₋₁, RHRₙ₋₁, KₙP, RₙP, KPKₙ₋₁, or RPRₙ₋₁, wherein n=1, 2, 3, 4, 5, 6, 7, 8, 9, or 10.

10. The composition of claim 9 wherein the peptide comprises KWK, KWK₂, K₂WK, KWK₄, or K₄WK.

11. The composition of claim 1 wherein the grafting density of the peptide to the hydrophobic block of the amphiphilic block copolymer is about 1 to about 10.

12. The composition of claim 11 wherein the grafting density of the peptide to the hydrophobic block of the amphiphilic block copolymer is about 4, about 8, or about 12.

13. The composition of claim 1 wherein the polynucleotide encodes a protein product.

14. The composition of claim 1 wherein the one or more isolated polynucleotides condense into a compact, ordered DNA condensate.

15. The composition of claim 14, wherein the DNA condensate forms a rod structure, a toroid structure, and/or a spherical structure.

16. The composition of claim 1 further comprising a pharmaceutical carrier suitable for administration to a mammal for gene therapy.

17. Method of delivering one or more isolated polynucleotides to a cell, the method comprising contacting the cell with the composition of claim 1.

18. A polymer-peptide hybrid; wherein the polymer of the polymer-peptide hybrid comprises a neutral amphiphilic block copolymer;
wherein the peptide of the polymer-peptide hybrid is covalently attached to the hydrophobic block of the amphiphilic block copolymer; and
wherein the peptide of the polymer-peptide hybrid is a DNA binding peptide.

19. The polymer-peptide hybrid of claim 19, wherein the DNA binding peptide is selected from the group consisting of Kₙ, Rₙ, KₙWKₘ, RₙWHRₘ, KₙHKₘ, RₙHRₘ, KₙPKₘ, RₙPRₘ, wherein n=0, 1, 2, 3, 4, 5, 6, 7, 8, 9, or 10 and wherein m=0, 1, 2, 3, 4, 5, 6, 7, 8, 9, or 10.

20. The polymer-peptide hybrid of claim 19, wherein the polymer of the polymer-peptide hybrid comprises PBDₘ-b-PEOₙ, wherein n and m are independently about 8 to about 120.


SEQUENCE LISTING (8 sequences)

SEQ ID NO: 1 - DNA, 175 bp, artificial
  A-track sequence
atccatcgaccaaaaaacgggcaaaaaacggcaaaaaacgggcaaaaaacggcaaaaaacgggcaaaaaacggcaaaaaacgggcaaaaaacggcaaaaaacgggcaaaaaacggcaaaaaacgggcaaaaaacggcaaaaaacgggcaaaaaacggcaaaaaagcagtggaagc

SEQ ID NO: 2 - DNA, 20 bp, artificial
  synthetic oligonucleotide for condensation studies
cgcgcgcgcgcgcgcgcgcg

SEQ ID NO: 3 - DNA, 20 bp, artificial
  synthetic oligonucleotide for condensation studies
atatatatatatatatatat

SEQ ID NO: 4 - DNA, 20 bp, artificial
  synthetic oligonucleotide for condensation studies
cccccccccccccccccccc

SEQ ID NO: 5 - DNA, 20 bp, artificial
  synthetic oligonucleotide for condensation studies
gggggggggggggggggggg

SEQ ID NO: 6 - DNA, 20 bp, artificial
  synthetic oligonucleotide for condensation studies
aaaaaaaaaaaaaaaaaaaa

SEQ ID NO: 7 - DNA, 20 bp, artificial
  synthetic oligonucleotide for condensation studies
tttttttttttttttttttt

SEQ ID NO: 8 - DNA, 15 bp, artificial
  synthetic oligonucleotide complementary to ribosome-binding siteof c-Has-ras mRNA
atattccgtcatcgc